(19) United States
(12) Patent Application Publication
(10) Pub. No.: US 2025/0249165 A1
(43) Pub. Date: Aug. 7, 2025

(54) EMBOLIC PROTECTION SYSTEM AND RELATED METHODS

(71) Applicant: NeuroFine Corp., Miramar, FL (US)

(72) Inventors: Brad D. Aurilia, Coral Springs, FL (US); Ajay K. Wakhloo, Weston, MA (US)

(21) Appl. No.: 19/188,328

(22) Filed: Apr. 24, 2025

Related U.S. Application Data
(63) Continuation of application No. PCT/US2023/036651, filed on Nov. 2, 2023.
(60) Provisional application No. 63/431,366, filed on Dec. 9, 2022.
(60) Provisional application No. 63/422,345, filed on Nov. 3, 2022.

(51) Int. Cl. A61M 1/36; A61B 17/00; A61B 17/06; A61B 17/12; A61M 39/22

(52) U.S. Cl. CPC A61M 1/3655; A61B 17/0057; A61B 17/06066; A61B 17/12031; A61B 17/12109; A61B 17/12136; A61M 1/362264; A61M 39/223; A61B 2017/0061; A61B 2017/00632; A61B 2017/00663; A61B 2017/00778

(57) ABSTRACT

An embolic protection system that includes a vessel access device, a vessel return device, a filter, tubing, and a vessel closure device. The vessel access device is configured to percutaneously access a first vessel. The vessel return device is configured to access a second vessel. The filter is configured to collect emboli. The tubing is configured to couple the vessel access device, the vessel return device, and the filter. The embolic protection system is configured to direct blood flow from the first vessel, through the vessel access device, the filter, and the vessel return device, and into the second vessel. The vessel closure device is configured to percutaneously close an opening in the first vessel.

### CROSS REFERENCE TO RELATED APPLICATIONS

This application is a continuation of PCT/US2023/036651 filed on Nov. 2, 2023 (pending), which claims the priority of U.S. Provisional Patent Application No. 63/422,345 filed on Nov. 3, 2022 (expired) and U.S. Provisional Application Ser. No. 63/431,366 filed on Dec. 9, 2022 (expired), the disclosures of which are incorporated herein by reference in their entirety.

This application is also related to U.S. patent application Ser. No. 18/669,040 filed on May 20, 2024 (pending), U.S. patent application Ser. No. 18/751,896 filed on Jun. 24, 2024 (pending), U.S. patent application Ser. No. 17/546,947 filed on Dec. 9, 2021 (now U.S. Pat. No. 12,035,905) and U.S. patent application Ser. No. 17/546,958 filed on Dec. 9, 2021 (now U.S. Pat. No. 12,048,429), the disclosures of which are incorporated herein by reference in their entirety.

### TECHNICAL FIELD

Embodiments relate generally to embolic protection devices, systems, and related methods that directly-access blood vessels, provide embolic protection during therapy, and close punctures or other openings in blood vessels.

### BACKGROUND

Vascular access for neuro-therapy includes the risk of stroke and other complications due to emboli (loose arterial plaque) that may be generated during a procedure. One of the current methods of providing embolic protection includes placing temporary filters inside a neurovascular artery to collect emboli that may be generated during a common carotid artery (CCA) vascular stenting procedure, for example. The filters are removed at the end of the procedure or at some time post procedure.

Another method of providing embolic protection, and the preferred approach, includes creating retrograde carotid artery blood flow (flow reversal) thereby flowing blood from the cerebral vessels down the internal carotid artery and external carotid artery (ICA/ECA respectively) into the common carotid artery where the blood flow exits the patient via an introducer sheath thus allowing any generated emboli (loose arterial plaque) to flow away from the cerebrum and be filtered out of the bloodstream prior to being re-introduced into the patient via a femoral vein.

While the preferred approach to embolic protection is the blood flow reversal method, blood flow reversal devices currently on the market do not utilize the direct carotid artery puncture approach due to an inability to percutaneously close the artery puncture site.

Vascular access for neuro-therapy is traditionally performed through the femoral artery or by using a radial access approach. For vascular access to the brain using a traditional approach, neuro-therapy devices must navigate through lengthy tortuous segments of the anatomy to gain access to the therapeutic site. The Direct Carotid Artery Puncture (DCP) method of accessing the CCA allows doctors quicker access to the brain and eliminates the need for devices that must traverse the typical femoral interventional track. The DCP method includes a percutaneous puncture in the skin and arterial vessel to access the CCA.

Currently, to close and seal a direct carotid artery puncture, doctors may apply a suture manually prior to accessing the common carotid artery, and the suture is used to close the puncture post procedure. Alternatively, direct pressure may be applied to the site after intervention until the vessel seals itself. Applying direct pressure to the site relies on blood coagulation at the puncture site and may be ineffective because of the lack of suitable anatomy in the vicinity of the common carotid artery against which the pressure may be applied. The time period necessary for closing the puncture with direct pressure may also be quite lengthy. The common carotid artery carries blood at a high pressure (100 to 200 mmHg) which further complicates the ability to effectively close the carotid puncture with direct pressure or other current methods. While the DCP method provides doctors quicker access to the brain, current methods of arterial puncture closure present challenges.

Accordingly, and despite the various advances already made in this field, there is a need for further improvements related to devices, systems and methods for accessing blood vessels, providing embolic protection, and sealing punctures or other openings in blood vessels.

### SUMMARY

Generally, an embolic protection system is provided herein. The embolic protection system includes a vessel access device, a vessel return device, a filter, tubing, and a vessel closure device. The vessel access device is configured to percutaneously access a first vessel. The vessel return device is configured to access a second vessel. The filter is configured to collect emboli. The tubing is configured to couple the vessel access device, the vessel return device, and the filter. The vessel closure device is configured to percutaneously close an opening in the first vessel. The system is configured to direct blood flow from the first vessel, through the vessel access device, the filter, and the vessel return device, and into the second vessel.

In some embodiments, the vessel closure device may be configured to access the first vessel through the vessel access device. The system may include a flow control device configured to be coupled to the vessel access device, the vessel return device, and the filter with the tubing and may be configured to control the blood flow from the first vessel to the second vessel.

In some embodiments, the vessel access device may include a vessel sealing portion configured to seal the vessel access device at least partially against the inside of the first vessel when the vessel sealing portion is activated. The vessel sealing portion may include an expandable element and activating the vessel sealing portion may include expanding the expandable element of the vessel sealing portion thereby at least partially sealing the vessel access device against the inside of the first vessel. In alternative embodiments, the expandable element may include a balloon and inflating the balloon may seal the vessel access device at least partially against the inside of the first vessel. In other embodiments, the vessel sealing portion may include a flexible material and activating the vessel sealing portion may include compressing the flexible material to radially expand the flexible material thereby sealing the vessel access device at least partially against the inside of the first vessel. The vessel sealing portion may include a pre-formed structure and activating the vessel sealing portion may allow the pre-formed structure to expand radially thereby sealing the vessel access device at least partially against the inside of the first vessel. The pre-formed structure may include a wire structure. The wire structure may include at least one of a film or a coating that at least partially covers the wire structure to prevent blood flow through the wire structure. The vessel access device may include a seal actuator configured to activate and/or deactivate the vessel sealing portion. Moving the seal actuator axially may activate and/or deactivate the vessel sealing portion.

In some embodiments, the vessel access device may be an introducer sheath. The vessel return device may be an introducer sheath. The vessel access device may include a radiopaque marker. The vessel closure device may be configured to percutaneously close an opening in the second vessel. The vessel closure device may be configured to access the second vessel through the vessel return device.

An alternative embolic protection system is also disclosed. The alternative embolic protection system includes a vessel access device, a vessel return device, a filter, a flow control device, tubing, and a vessel closure device. The vessel access device is configured to percutaneously access a first vessel. The vessel access device includes a vessel sealing portion configured to seal the vessel access device at least partially against the inside of the first vessel when the vessel sealing portion is activated. The vessel return device configured to access a second vessel. The filter is configured to collect emboli. The tubing is configured to couple the vessel access device, the vessel return device, the filter, and the flow control device. The vessel closure device is configured to percutaneously close an opening in the first vessel. The embolic protection system is configured to direct blood flow from the first vessel, through the vessel access device, the filter, the flow control device, and the vessel return device, and into the second vessel. The flow control device is configured to control the blood flow from the first vessel to the second vessel.

In some embodiments, the vessel closure device may be configured to access the first vessel through the vessel access device. The vessel sealing portion may include an expandable element. Activating the vessel sealing portion may include expanding the expandable element of the vessel sealing portion thereby sealing the vessel access device at least partially against the inside of the first vessel. The expandable portion may include a balloon and inflating the balloon seals the vessel access device at least partially against the inside of the first vessel. The sealing portion may include a flexible material and activating the vessel sealing portion may include compressing the flexible material to radially expand the flexible material thereby sealing the vessel access device at least partially against the inside of the first vessel. The sealing portion may include a pre-formed structure and activating the vessel sealing portion may allow the pre-formed structure to expand radially thereby sealing the vessel access device at least partially against the inside of the first vessel. The pre-formed structure may include a wire structure. The wire structure may include at least one of a film or a coating that at least partially covers the wire structure to prevent blood flow through the wire structure. The vessel access device may include a seal actuator configured to activate and/or deactivate the vessel sealing portion. Moving the seal actuator axially may activate and/or deactivate the vessel sealing portion.

In some embodiments, the vessel access device may be an introducer sheath. The vessel return device may be an introducer sheath. The vessel access device may include a radiopaque marker. The vessel closure device may be configured to percutaneously close an opening in the second vessel. The vessel closure device may be configured to access the second vessel through the vessel return device.

A method of providing embolic protection is provided herein. The method of providing embolic protection includes percutaneously accessing a first vessel with a vessel access device, accessing a second vessel with a vessel return device, coupling a filter and tubing to the vessel access device and the vessel return device. The method includes establishing blood flow from the first vessel, through the vessel access device, the filter, and the vessel return device, and into the second vessel. The method also includes percutaneously closing an opening in the first vessel.

The method of providing embolic protection may include coupling a flow control device to the tubing, directing the blood flow through the flow control device, and controlling the blood flow from the first vessel to the second vessel with the flow control device. The first vessel may be a carotid artery. The second vessel may be a femoral vein. Accessing the second vessel may include percutaneously accessing the second vessel with the vessel return device. The method may include percutaneously closing an opening in the second vessel. The method may include sealing the vessel access device at least partially against the inside of the first vessel. Sealing the vessel access device at least partially against an inside of the first vessel may include expanding a vessel sealing portion of the vessel access device. The vessel sealing portion of the vessel access device may include a balloon and the method may include inflating the balloon. Sealing the vessel access device at least partially against the inside of the first vessel further may include compressing a flexible material of a vessel sealing portion of the vessel access device to radially expand the flexible material. Sealing the vessel access device at least partially against the inside of the first vessel may include activating a pre-formed structure of a sealing portion of the vessel access device allowing the pre-formed structure to expand radially. Sealing the vessel access device at least partially against the inside of the first vessel may include activating and/or deactivating a vessel sealing portion of the vessel access device with a seal actuator. The method may include moving the seal actuator axially to activate and/or deactivate the vessel sealing portion.

### BRIEF DESCRIPTION OF DRAWINGS

FIG. 1 is a schematic diagram of an illustrative embolic protection system.

FIG. 2 is a side view of an illustrative vessel access device of the embolic protection system of FIG. 1.

FIG. 2A is a sectional view of the vessel access device of FIG. 2.

FIG. 2B is a side view of the vessel access device of FIG. 2 with a vessel sealing portion activated.

FIG. 3 is a side view of an illustrative vessel return device of the embolic protection system of FIG. 1.

FIG. 3A is a sectional view of the vessel return device of FIG. 3.

FIG. 4 is a side view of an illustrative filter, flow control device, and 3-way valve of the embolic protection system of FIG. 1.

FIG. 5A depicts the vessel access device of FIG. 2 percutaneously inserted into a blood vessel.

FIG. 5B depicts the vessel access device of FIG. 2 sealed at least partially against the inside of a blood vessel.

FIG. 6 depicts the vessel return device of FIG. 3 percutaneously inserted into a blood vessel.

FIGS. 7A and 7B are sectional views of an alternative illustrative vessel access device.

FIGS. 8A and 8B are sectional views of another illustrative vessel access device.

FIG. 9 is a schematic diagram of an alternative illustrative embolic protection system.

FIG. 10A is a side view of an alternative illustrative vessel access device.

FIG. 10B is a sectional view of the vessel access device of FIG. 10A.

FIG. 11 depicts the vessel access device of FIG. 10A percutaneously inserted into a blood vessel.

FIG. 12 is a perspective view of an illustrative vessel closure device.

FIGS. 13A, 13B, and 13C are perspective views of a suturing mechanism at the distal end of the vessel closure device of FIG. 12.

FIG. 14 shows the vessel closure device of FIG. 12 inserted through the vessel access device of FIG. 2.

FIGS. 15A through 15J are a progressive series of side views of the distal end of the vessel closure device of FIG. 12 during a suturing procedure.

FIG. 16 is a perspective view of an alternative illustrative vessel closure device.

FIG. 17 is an exploded sectional view of a handle of the vessel closure device of FIG. 16.

FIGS. 18A, 18B and 18C are perspective views of a suturing mechanism at the distal end of the vessel closure device of FIG. 16.

FIG. 19A is a top view of the handle of the vessel closure device of FIG. 16.

FIG. 19B is a section view of the suturing mechanism of the vessel closure device of FIG. 16.

FIG. 20A is a top view of the handle of the vessel closure device of FIG. 16.

FIG. 20B is a section view of the suturing mechanism of the vessel closure device of FIG. 16.

FIG. 21A is a section view of the handle of the vessel closure device of FIG. 16.

FIG. 21B is a section view of the suturing mechanism of the vessel closure device of FIG. 16.

FIG. 22A is a section view of the handle of the vessel closure device of FIG. 16.

FIG. 22B is a section view of the suturing mechanism of the vessel closure device of FIG. 16.

FIG. 23A is a section view of the handle of the vessel closure device of FIG. 16.

FIG. 23B is a section view of the suturing mechanism of the vessel closure device of FIG. 16.

FIG. 24A is a section view of the handle of the vessel closure device of FIG. 16.

FIG. 24B is a section view of the suturing mechanism of the vessel closure device of FIG. 16.

FIG. 25 is a perspective view of the handle of the vessel closure device of FIG. 16.

FIGS. 26A through 26J are a progressive series of side views of the distal end of the vessel closure device of FIG. 16 during a suturing procedure.

FIG. 27 is a top view of an alternative illustrative vessel closure device handle.

FIG. 28 is a top view an another illustrative vessel closure device handle.

### DETAILED DESCRIPTION

The present disclosure generally relates to devices, systems and methods for accessing blood vessels, providing embolic protection, and sealing punctures or other openings in blood vessels. An embolic protection device and/or system is described herein. The embolic protection system is described directly-accessing the common carotid artery (CCA), but may be used to access other blood vessels. The embolic protection system provides embolic protection during neurovascular therapy (neuro-therapy), such as Trans Carotid Artery Revascularization (TCAR), for example. The system also includes a suture device for percutaneously sealing openings in blood vessels, such as a carotid artery or a femoral vein. The devices, systems, and methods may be used in the course of other therapies or surgical procedures as well.

The direct carotid artery puncture (DCP), blood flow reversal, and closure system disclosed herein is intended, for example, to be used as an embolic protection device that can directly-access the common carotid artery and provide embolic protection by allowing for a retrograde blood flow from the common carotid artery to a femoral vein during neurovascular (neuro) therapy such as TCAR.

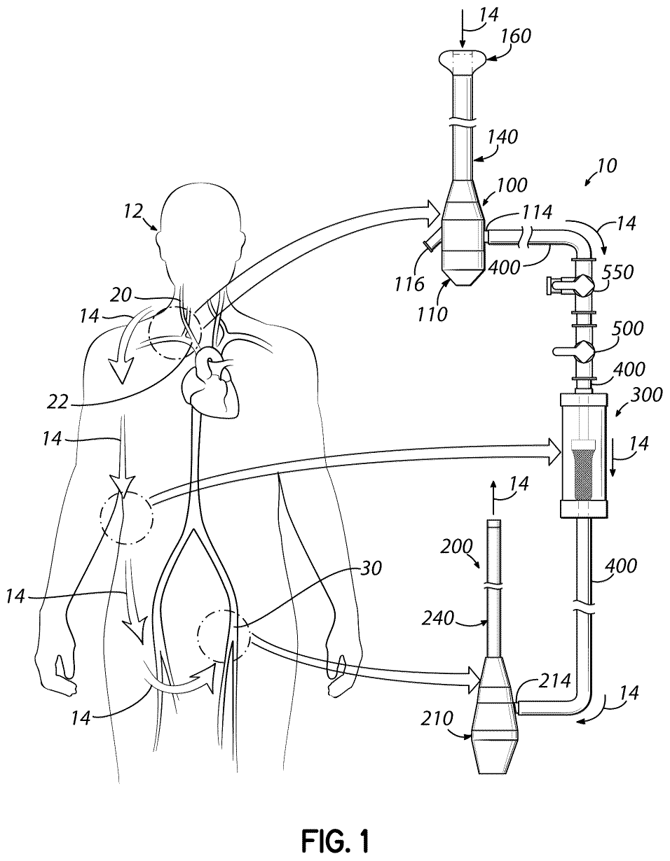

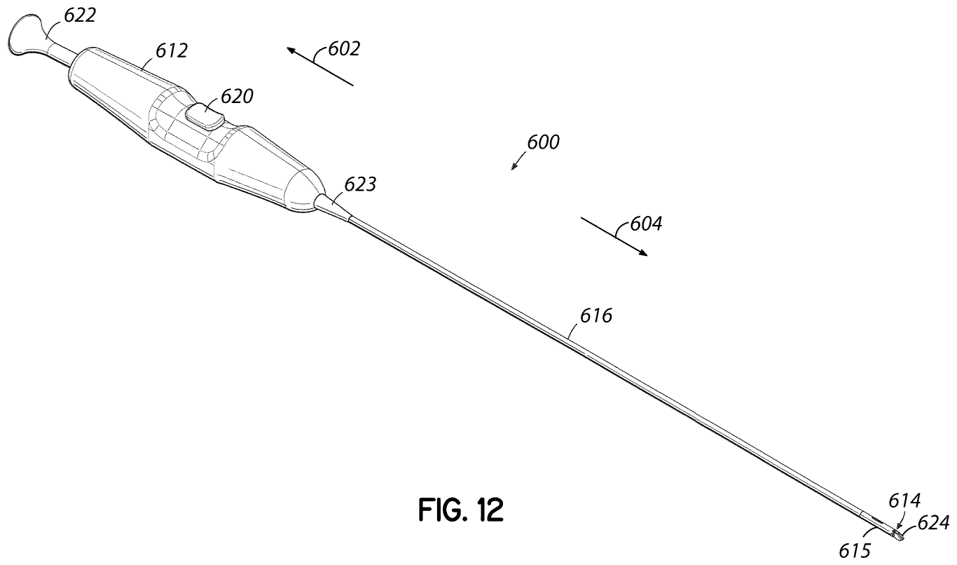

FIG. 1 is a schematic of an illustrative embolic protection system 10. In this illustrative example, when in use with a patient 12, the embolic protection system 10 allows for a retrograde blood flow from a patient's common carotid artery 20 to a femoral vein 30 with the direction of blood flow shown by arrows 14. In this illustrative embodiment, the embolic protection system 10 includes a vessel access device 100, a vessel return device 200, a filter 300, tubing 400, a flow control device 500, a 3-way valve 550, and a vessel closure device 600, see FIG. 12. As used herein, the term “vessel”, “blood vessel”, “arteries”, “veins”, and similar forms of these terms mean any portion of the circulatory system that transports blood throughout the human body.

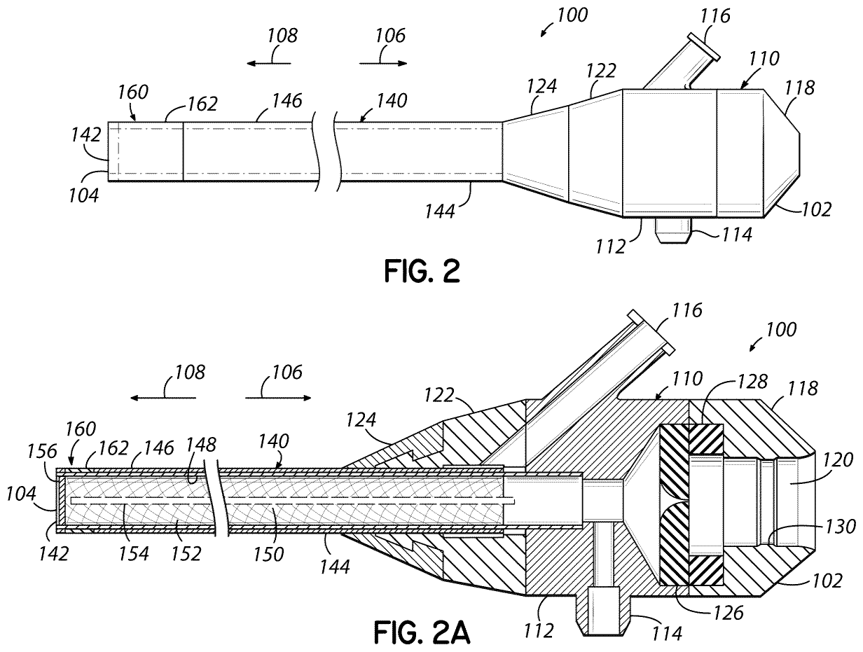

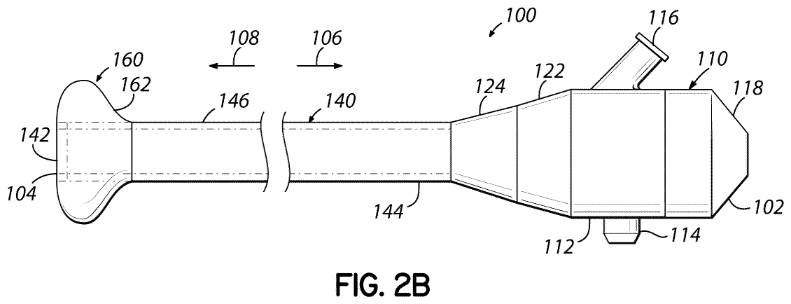

FIG. 2 is a side view of an illustrative vessel access device 100. FIG. 2A is a sectional view detailing the construction of the vessel access device 100. In this illustrative embodiment, the vessel access device 100 is an introducer sheath configured to percutaneously access a blood vessel, such as a carotid artery. The vessel access device 100 has a proximal end portion 102, a distal end portion 104, a hub 110, a sheath 140, and a vessel sealing portion 160.

As used herein to describe various embodiments from the perspective of a user of a surgical device, “proximal” may refer to a direction generally towards the user of the device, while “distal” may refer to a direction generally away from the user of the device. Similarly, in the context of a surgical device inserted into a patient's body and from the perspective of a user of the device, “proximal” may refer to a direction generally away from the patient's body, and “distal” may refer to a direction generally towards the patient's body. For reference, arrow 106 points generally proximally and arrow 108 points generally distally.

The hub 110 includes a body 112, a side port 114, an inflation port 116, a rear hub body 118, a central lumen 120, a front hub body 122, a strain relief 124, a dilator seal 126, a compression washer 128, and a lock 130. The outer surface of the hub 110 may include indentions or other surface features to improve the ergonomics of the hub 110. The side port 114 may be used to flush air from the vessel access device 100 at the start of a procedure, for example. In this embodiment, the side port 114 serves as a conduit for a retrograde blood flow during embolic protection as described herein.

Referring again to FIGS. 2 and 2A, the dilator seal 126 is designed to seal off blood flow without the need for an external valve such as a Tuohy Borst valve, for example. A user can access the central lumen 120 at the proximal end portion 102 of the hub 110 with guidewires, dilators, and other interventional devices, for example, through the dilator seal 126 without concern for bleed-back. The dilator seal 126 remains in a closed position when a device, such as a dilator for example, is not passing through the central lumen 120. The lock 130 located in the rear hub body 118 allows a device, such as a dilator for example, to lock into the hub 110 prior to or during a procedure, such as a vessel dilation for example. The lock 130 allows for the device, such as a dilator for example, to be easily removed from the vessel access device 100 when the procedure is complete.

The sheath 140 has a distal end portion 142 and a proximal end portion 144. The proximal end portion 144 of the sheath 140 passes through the strain relief 124 and is coupled to the hub 110. The sheath 140 also includes an outer jacket 146, an inner liner 148, a reinforcing portion 150, a central lumen 152, an inflation lumen 154, one or more radiopaque markers 156, and a vessel sealing portion. In this illustrative embodiment, the inner liner 148 provides a lubricious surface for the interventional devices to slide within. The reinforcing portion 150 includes cross coiled filaments and/or wire. The cross coiled filaments and/or wire of the reinforcing portion 150 provide flexibility and kink resistance to the sheath 140 while maintaining the tensile strength of the sheath 140. The sheath 140 needs to be flexible while also resistant to kinking during procedures requiring advancing the vessel access device 100 through tissue during a procedure, for example. A hydrophilic coating may be applied to the outer surface of the sheath to reduce the frictional forces during insertion and removal procedures. The central lumen 152 extends from the distal end portion 142 to the proximal end portion 144 of the sheath 140 and aligns with the central lumen 120 of the hub 110. A radiopaque marker 156 is located at the distal end portion 142 of the sheath 140. The radiopaque marker 156 is used during procedures in conjunction with a medical imaging device to assist a user in guiding the distal end portion 142 of the sheath 140 to a desired location within a blood vessel. Radiopaque markers 156 may be located at other locations on the sheath 140.

FIG. 2B is a perspective view of the illustrative vessel access device 100 showing the vessel sealing portion 160 activated. Referencing FIGS. 2, 2A, and 2B, the vessel access device 100 has a vessel sealing portion 160 located at the distal end portion 142 of the sheath 140. The vessel sealing portion 160 is configured to seal the distal end portion 142 of the sheath 140 at least partially against the inside of a blood vessel when the vessel sealing portion 160 is activated. In this illustrative embodiment, the vessel sealing portion 160 includes an expandable element 162. Activating the vessel sealing portion 160 includes expanding the expandable element 162 of the vessel sealing portion 160 thereby sealing the vessel access device 100 at least partially against the inside of a blood vessel. In this illustrative embodiment, the expandable element 162 is an inflatable element including a balloon. Inflating the balloon seals the vessel access device 100 at least partially against the inside of a blood vessel.

In this illustrative embodiment, the inflation lumen 154 is coupled to the inflation port 116 on the hub 110 and extends to and is coupled to the vessel sealing portion 160 at the distal end portion 142 of the sheath 140. While the vessel sealing portion 160 of the vessel access device 100 is inside a blood vessel, a user injects fluid into the inflation port 116 and the fluid will pass through the inflation lumen 154 and into the vessel sealing portion 160, thereby expanding and/or inflating the expandable portion 162, see FIG. 2B, and at least partially sealing the vessel access device 100 against the inside of a blood vessel. Withdrawing the fluid from the inflation port 116 will contract and/or deflate the expandable portion 162, see FIG. 2 thereby withdrawing the vessel sealing portion 160 from the inside of a blood vessel.

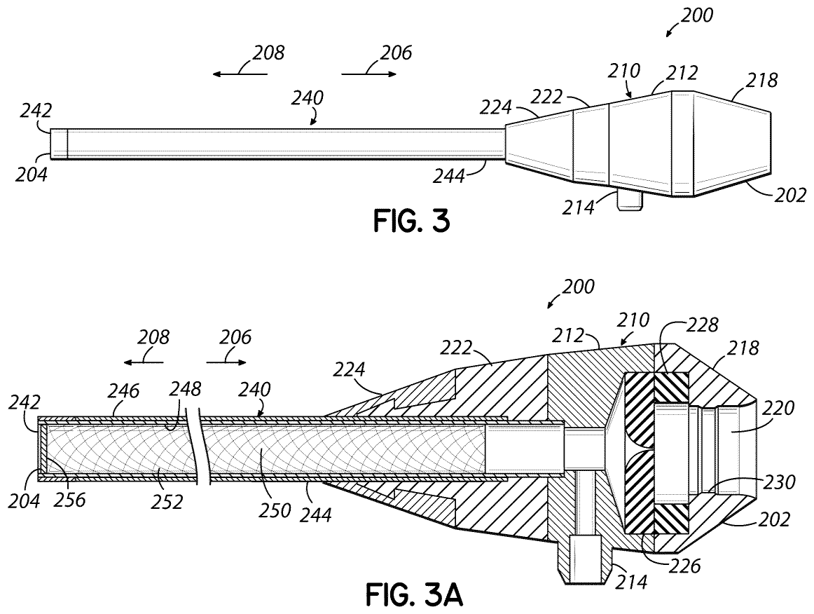

FIG. 3 is a perspective view of an illustrative vessel return device 200. FIG. 3A is a sectional view detailing the construction of the vessel return device 200. In this illustrative embodiment, the vessel return device 200 is an introducer sheath configured to percutaneously access a blood vessel, such as a femoral vein. The vessel return device 200 has a proximal end portion 202, a distal end portion 204, a hub 210 and a sheath 240. For reference, arrow 206 points generally proximally and arrow 208 points generally distally. The hub 210 includes a body 212, a side port 214, a rear hub body 218, a central lumen 220, a front hub body 222, a strain relief 224, a dilator seal 226, and a compression washer 228. The outer surface of the hub 210 may include indentions or other surface features to improve the ergonomics of the hub 210.

The dilator seal 226 is designed to seal off blood flow without the need for an external valve such as a Tuohy Borst valve, for example. A user can access the proximal end portion 202 of the hub 210 with guidewires, dilators, and interventional devices, for example, through the dilator seal 226 without concern for bleed-back. The dilator seal 226 remains in a closed position when a device, such as a dilator for example, is not passing through the central lumen 220. The rear hub body 218 includes a lock 230 allowing a device, such as a dilator for example, to lock into the hub 210 prior to or during a procedure, such as a vessel dilation for example. The lock 230 allows for the device, such as a dilator for example, to be easily removed from the vessel return device 200 when the procedure is complete. The side port 214 may be used to flush air from the vessel return device 200 at the start of a procedure. The side port 214 serves as a conduit for a retrograde blood flow during embolic protection as described herein.

The sheath 240 has a distal end portion 242 and a proximal end portion 244. The proximal end portion 244 of the sheath 240 passes through the strain relief 224 and is coupled to the hub 210. The sheath 240 also includes an outer jacket 246, an inner liner 248, a reinforcing portion 250, a central lumen 252, and one or more radiopaque markers 256. In this illustrative embodiment, the reinforcing portion 250 includes cross coiled filaments and/or wire which provide flexibility and kink resistance to the sheath 240 while maintaining the tensile strength of the sheath 240. The sheath 240 needs to be flexible while also resistant to kinking during procedures requiring advancing the vessel return device 200 through tissue during a procedure, for example. The central lumen 252 extends from the distal end portion 242 to the proximal end portion 244 of the sheath 240 and aligns with the central lumen 220 of the hub 210. A radiopaque marker 256 is located at the distal end portion 242 of the sheath 240. The radiopaque marker 256 is used during a procedure in conjunction with a medical imaging device to assist a user in guiding the distal end portion 242 of the sheath 240 to a desired location within a blood vessel. Radiopaque markers 256 may be located at other locations on the sheath 250.

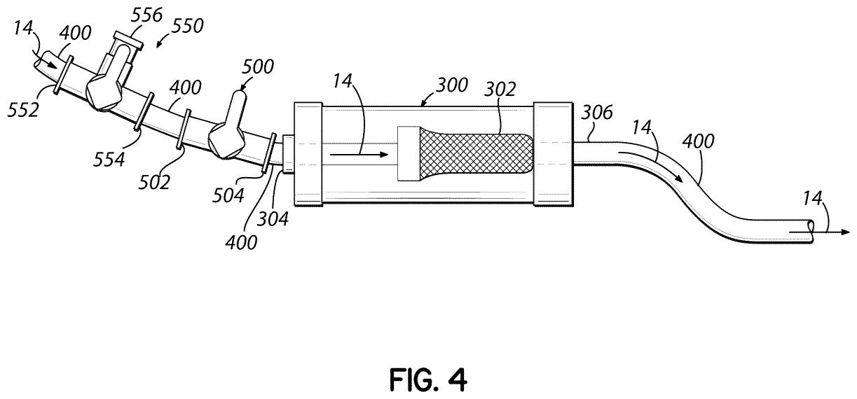

FIG. 4 is a side view of an illustrative filter 300, flow control device 500, and 3-way valve 550 of the embolic protection system 10. In this illustrative embodiment, the filter 300 is configured to filter blood and collect emboli (plaque) that is generated during a procedure, such as a neurovascular procedure, for example. The filter 300 has a filter element 302, an inlet 304, and an outlet 306. The filter element 302 is configured to filter blood and collect emboli to provide embolic protection while allowing sufficient blood flow during a procedure. As indicated by arrows 14, when the filter 300 is in use, blood flows into the filter 300 though the inlet 304, though the filter element 302, and out through the outlet 306.

In this illustrative embodiment, the flow control device 500 has an inlet 502, and an outlet 504. As indicated by arrows 14, when the flow control device 500 is in use, blood flows into the flow control device 500 though the inlet 502, though the flow control device 500, and out through the outlet 504. The flow control device 500 is configured to control the flow of blood. In some embodiments, the flow control device 500 may be adjustable by a user, for example, while in alternative embodiments, the flow control device 500 may automatically adjust the blood flow. In alternative embodiments, the flow control device 500 may be used to reduce the flow of blood during a procedure, for example. In some embodiments, the flow control device 500 may be used to start or stop the flow of blood, such as at the beginning and/or end of procedure, for example. In some embodiments, the flow control device 500 may be a stopcock valve (2-way or 3-way) while in alternative embodiments, the flow control device 500 may be another type of valve and/or flow restrictor. In some embodiments, the flow control device 500 may be coupled proximally to the filter 300 while in other embodiments, the flow control device 500 may be integral to the filter 300 as part of a filter assembly, for example.

In this illustrative embodiment, the 3-way valve 550 has an inlet 552, an outlet 554, and an access port 556. As indicated by arrows 14, when the 3-way valve 550 is in use, blood flows into the 3-way valve 550 though the inlet 552, though the 3-way valve 550, and out through the outlet 554. The 3-way valve 550 is configured to give a user an access port 556 for injecting saline or contrast into the patient at the start of or during a procedure. The 3-way valve 550 also allows for removal of air from the embolic protection system 10 at the start of a procedure, such as a retrograde blood flow procedure, for example. In some embodiments, the 3-way valve 550 may be combined with the filter 300 and/or the flow control device 500.

Referring to FIGS. 1 and 4, in this illustrative embodiment, the tubing 400 couples the vessel access device 100, the vessel return device 200, the filter 300, the 3-way valve 550, and the flow control device 500. As shown by the arrows 14 the tubing 400 is configured to direct a blood flow from a common carotid artery 20, through the vessel access device 100, the filter 300, the 3-way valve 550, the flow control device 500, and the vessel return device 200, and into a femoral vein 30.

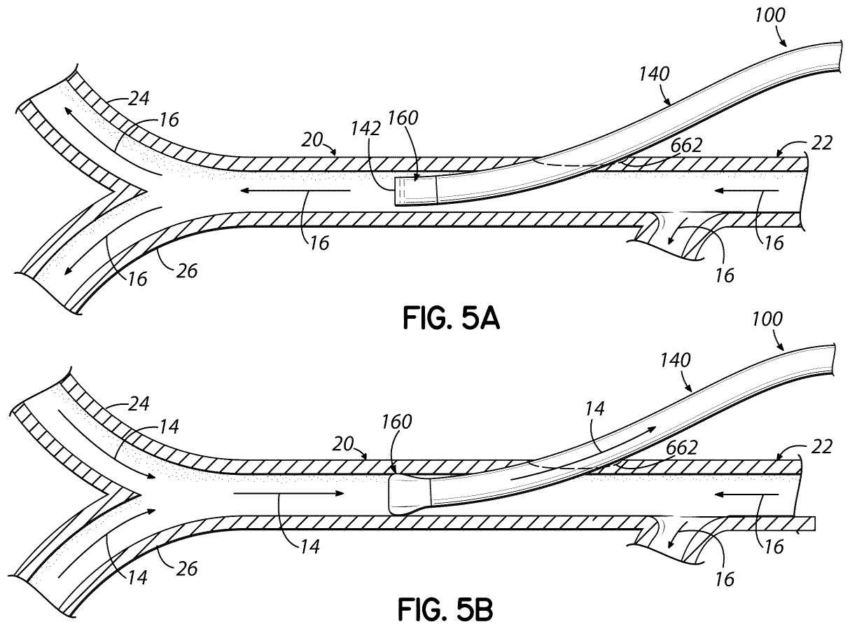

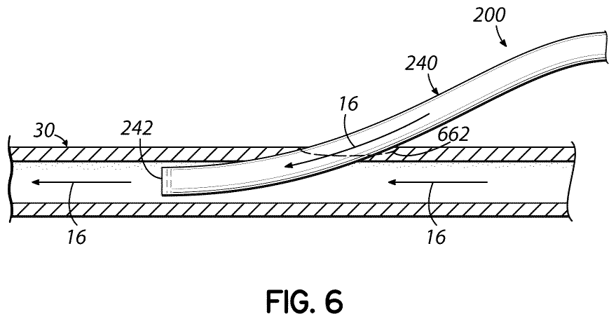

FIGS. 1, 5A, 5B, and 6 illustrate a method of percutaneously accessing blood vessels and establishing a retrograde blood flow to provide embolic protection while another medical procedure is performed, such as a neurovascular procedure, is described herein. In this example, the blood vessels being accessed for establishing retrograde blood flow are a common carotid artery 20 and a femoral vein 30. FIG. 5A depicts the vessel access device 100 percutaneously inserted into the common carotid artery 20 with the sheath 140 aligned with the lumen of the common carotid artery 20. FIG. 5B depicts the vessel access device 100 sealed at least partially against the inside of the common carotid artery 20. FIG. 6 depicts the vessel return device 200 percutaneously inserted into a femoral vein 30.

A common carotid artery 20, and a suitable location for percutaneously accessing the common carotid artery 20, are identified using ultrasound or other non-invasive methods. Using ultrasound, or other suitable methods, a needle and guidewire are directed to the identified access location on the common carotid artery 20. The common carotid artery 20 is pierced with the needle and the guidewire is inserted into the common carotid artery 20. A dilator is inserted into the vessel access device 100 and directed along the guidewire to create an opening in the common carotid artery 20. The dilator is used to dilate the opening in the common carotid artery 20 so the distal end portion 142 of the sheath 140 of the vessel access device 100 can access the inside of the common carotid artery 20.

The vessel access device 100 is percutaneously inserted into the common carotid artery 20 with the sheath 140 aligned with the lumen of the common carotid artery 20, see FIG. 5A. Fluid is injected into the inflation port 116 of the hub 110, expanding the expandable element 162 of the vessel sealing portion 160 of the vessel access device 100, see FIG. 5B. The vessel sealing portion 160 is expanded so that the vessel sealing portion 160 is engaged with the inside of the common carotid artery 20 thereby at least partially sealing the vessel access device 100 against the inside of the common carotid artery 20. In this example, sealing the vessel access device 100 at least partially to the inside of the common carotid artery 20 temporarily blocks the anterograde blood flow, indicated by arrow 16 in FIG. 5A, from the brachiocephalic artery 22 to the common carotid artery 20.

Similar to the method of accessing the common carotid artery 20 described above, in this example, a femoral vein 30, and a suitable location for percutaneously accessing the femoral vein 30 are identified using ultrasound or other non-invasive methods. Using ultrasound, or other suitable methods, a needle and guidewire are directed to the access location on the femoral vein 30. The femoral vein 30 is pierced with the needle and the guidewire is inserted into the femoral vein 30. A dilator is inserted into the vessel return device 200 and directed along the guidewire to create an opening in the femoral vein 30. The dilator is used to dilate the opening in the femoral vein 30 so the distal end portion 242 of the sheath 240 of the vessel return device 200 can access the inside of the femoral vein 30. The vessel return device 200 is percutaneously inserted into the femoral vein 30 with the sheath 240 aligned with the lumen of the femoral vein 30, see FIG. 6.

Referencing FIGS. 1 and 4, in this illustrative example, tubing 400 is coupled to the side port 114 of the vessel access device 100 and the inlet 552 of the 3-way valve 550. Tubing 400 is coupled to the outlet 554 of the 3-way valve 550 and the inlet 502 of the flow control device 500. Tubing 400 is connected to the outlet 504 of the flow control device 500 and the inlet 304 of the filter 300. Tubing 400 is connected to the outlet 306 of the filter 300 and the side port 214 of the vessel return device 200. Alternatively, the filter 300, flow control device 500, and 3-way valve 550 may be connected in any order between the vessel access device 100 and the vessel return device 200. The filter 300, flow control device 500, and/or 3-way valve 550 may be connected to the vessel access device 100 and/or the vessel return device 200 before the vessel access device 100 is inserted into the common carotid artery 20 and/or the vessel return device 200 is inserted into the femoral artery 30.

Referring to FIGS. 1 and 4, in this illustrative example, a retrograde blood flow, identified by arrows 14, is established after the vessel access device 100 is sealed at least partially against the inside of the common carotid artery 20, the vessel return device 200 has accessed the femoral vein 30, and tubing 400 connects the vessel access device 100, 3-way valve 550, flow control device 500, filter 300, and vessel return device 200. At least partially sealing the vessel access device 100 to the inside of the common carotid artery 20 also temporarily blocks the anterograde blood flow, indicated by arrow 16, from the brachiocephalic artery 22 to the common carotid artery 20. Establishing a retrograde blood flow includes directing a retrograde blood flow from the internal carotid artery 24 and external carotid artery 26 to the common carotid artery 20. The retrograde blood flow is directed into the sheath 140 and out through the side port 114 of the vessel access device 100, through the tubing 400, 3-way valve 550, flow control device 500, filter 300, and into the side port 214 and out of the sheath 240 of the vessel return device 200. The retrograde blood flow is finally directed from the vessel return device 200 into the femoral vein 30. The blood pressure in the common carotid artery 20 is higher than the blood pressure in the femoral vein 30, and it is this pressure differential between the common carotid artery 20 and the femoral vein 30 that allows the retrograde blood flow from common carotid artery 20 to the femoral vein 30.

In this illustrative embodiment, the blood flow from the carotid artery 20 to the femoral vein 30 is controlled by adjusting and/or operating the flow control device 500. In some embodiments, the blood flow from the carotid artery 20 to the femoral vein 30 may be controlled automatically by the flow control device 500. In some cases, if the retrograde blood flow is too high for a patient, the patient could become disoriented (dizzy) or unconscious (black-out) due to the flow of blood away from the patient's brain. The flow control device 500 may be used to reduce the flow of blood to prevent the disorientation or loss of consciousness of the patient. During some medical procedures, the flow control device 500 may be used to reduce and/or stop the flow of blood to prevent other clinical situations.

The filter 300 captures any emboli (plaque) that are generated during a procedure, such as a neurovascular procedure for example. Once retrograde blood flow has been established, a neurovascular procedure can be performed with embolic protection provided by the filter 300. Procedures may include TCAR (Trans Carotid Artery Revascularization), Thrombectomy, Aneurism Coiling, etc.

After the procedure is complete, antegrade blood flow is restored. The flow control device 500 may be used to stop the retrograde blood flow. In this illustrative embodiment, fluid is withdrawn from the inflation port 116 thereby contracting and/or deflating the expandable portion 162 of the vessel sealing portion 160 of the vessel access device 100, see FIG. 5A. Contracting and/or deflating the expandable portion 162 withdraws the vessel sealing portion 160 from the inside of a blood vessel allowing antegrade blood flow, indicated by arrows 16, to be restored.

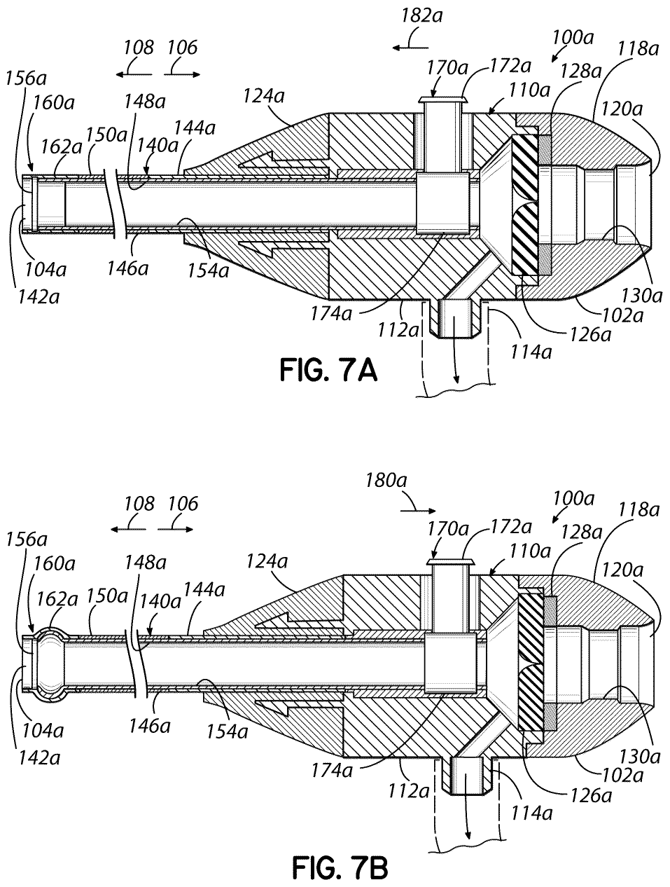

FIGS. 7A and 7B are sectional views detailing the construction of an alternative vessel access device 100a. Generally, the vessel access device 100a is similar in construction and operation to the vessel access device 100 described above and the vessel access device 100a may be substituted for other vessel access devices, or any feature of the vessel access device 100a may be used, in various other embodiments according to the present disclosure. Like reference numbers refer to like components. For brevity, the following description minimizes redundant description and focuses on the differences between the vessel access device 100a and the vessel access device 100.

In this illustrative embodiment, the vessel access device 100a has a proximal end portion 102a, a distal end portion 104a, a hub 110a, a sheath 140a, and a vessel sealing portion 160a. For reference, arrow 106 points generally proximally and arrow 108 points generally distally. The hub 110a includes a front hub body 112a, a side port 114a, a rear hub body 118a, a central lumen 120a, a strain relief 124a, a dilator seal 126a, a compression washer 128a, a lock 130a, and a seal actuator 170a.

The sheath 140a has a distal end portion 142a and a proximal end portion 144a. The sheath 140a also includes an outer jacket 146a, an inner liner 148a, a reinforcing portion 150a, a central lumen 152a, an activation member 154a, and one or more radiopaque markers 156a.

The vessel sealing portion 160a is located at the distal end portion 142a of the sheath 140a. The vessel sealing portion 160a is configured to seal the distal end portion 142a of the sheath 140a of the vessel access device 100a at least partially against the inside of a blood vessel, such as a carotid artery, when the vessel sealing portion 160a is activated. In this illustrative embodiment, the vessel sealing portion 160a is a flexible material 162a. The flexible material 162a may include silicone, urethane, or other similarly suitable and flexible material. The activation member 154a is coupled to the vessel sealing portion 160a at the distal end portion 142a of the sheath 140a and the seal actuator 170a at the hub 110a.

The seal actuator 170a includes a handle 172a coupled to a follower 174a which is coupled to a proximal end of the activation member 154a. The follower 174a is configured to move within the front hub body 112a of the hub 110a along a central axis of the vessel access device 100a. Moving the seal actuator 170a axially in the proximal direction, shown by arrows 106 and 180a, moves the activation member 154a in the proximal direction and activates the vessel sealing portion 160a. Activating the vessel sealing portion 160a includes axially compressing the flexible material 162a which causes the flexible material 162a to expand radially (circumferentially), see FIG. 7B, thereby sealing the vessel access device 100a at least partially against the inside of a blood vessel. Moving the seal actuator 170a axially in the distal direction, shown by arrows 108 and 182a, moves the activation member 154a in the distal direction and deactivates the vessel sealing portion 160a, see FIG. 7A. Deactivating the vessel sealing portion 160a includes axially decompressing the flexible material 162a causing the flexible material 162a to retract radially thereby withdrawing the vessel sealing portion 160a from the inside of a blood vessel. In this illustrative example, the seal actuator 170a includes a handle 172a. In other embodiments, the seal actuator 170a may include a knob, a lever, a button, or any other device or structure configured to move follower 174a and activation member 154a to activate and/or deactivate the vessel sealing portion 160a.

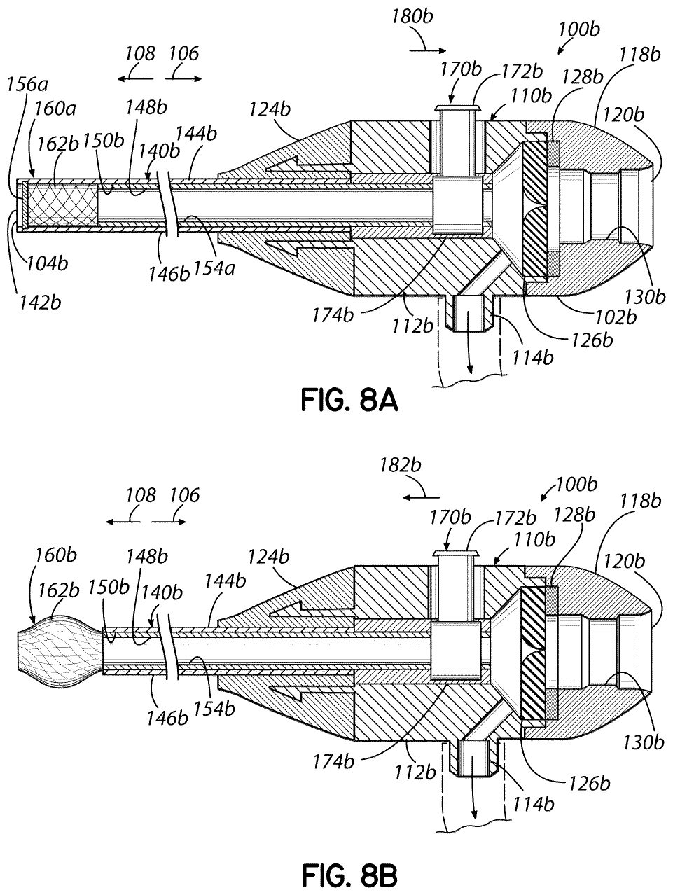

FIGS. 8A and 8B are sectional views detailing the construction of an alternative vessel access device 100b. Generally, the vessel access device 100b is similar in construction and operation to the vessel access devices 100 and 100a described above and the vessel access device 100b may be substituted for other vessel access devices, or any feature of the vessel access device 100b may be used, in various other embodiments according to the present disclosure. Like reference numbers refer to like components. For brevity, the following description minimizes redundant description and focuses on the differences between the vessel access device 100b and the vessel access devices 100 and 100a.

In this illustrative embodiment, the vessel access device 100b has a proximal end portion 102b, a distal end portion 104b, a hub 110b a sheath 140b, and a vessel sealing portion 160b. For reference, arrow 106 points generally proximally and arrow 108 points generally distally. The hub 110b includes a front body 112b, a side port 114b, a rear hub body 118b, a central lumen 120b, a strain relief 124b, a dilator seal 126b, a compression washer 128b, a lock 130b, and a seal actuator 170b.

The sheath 140b has a distal end portion 142b and a proximal end portion 144b. The sheath 140b also includes an outer jacket 146b, an inner liner 148b, a reinforcing portion 150b, a central lumen 152b, an activation member 154b and one or more radiopaque markers 156b.

The vessel access device 100b has a vessel sealing portion 160b located at the distal end portion 142b of the sheath 140b. The vessel sealing portion 160b is configured to seal the distal end portion 142b of the sheath 140b of the vessel access device 100b at least partially against the inside of a blood vessel, when the vessel sealing portion 160b is activated. In this illustrative embodiment, the vessel sealing portion 160b is a pre-formed structure 162b. As shown in FIG. 8A, the pre-formed structure 162b is located inside the sheath 140b in a deactivated or compressed state. As shown in FIG. 8B, the pre-formed structure 162b is located outside the sheath 140b when in an activated or expanded state. In this illustrative embodiment, the pre-formed structure 162b includes a pre-formed wire structure. In some embodiments, the wire structure may include a film or coating that at least partially covers the pre-formed structure 162b to prevent blood flow through the wire structure of the pre-formed structure 162b.

The seal actuator 170b includes a handle 172b coupled to a follower 174b which is coupled to a proximal end of the activation member 154b. The follower 174b is configured to move within the body 112b of the hub 110b along a central axis of the vessel access device 100b. Moving the seal actuator 170b axially in the distal direction, shown by arrows 108 and 182b, moves the activation member 154b in the distal direction thereby moving the pre-formed structure 162b out of the distal end 142b of the sheath 140b. Once outside the sheath 140b, the pre-formed structure 162b is activated and expands radially (circumferentially), see FIG. 8B, thereby sealing the vessel access device 100b at least partially against the inside of a blood vessel. Moving the seal actuator 170b proximally in the proximal direction, shown by arrows 106 and 180b, moves the outer jacket 146b in the distal direction thereby moving the pre-formed structure 162b inside the sheath 140b and deactivating the vessel sealing portion 160b, see FIG. 8A. In this illustrative example, the seal actuator 170b includes a handle 172b. In other embodiments, the seal actuator 170b may include a knob, a lever, a button, or any other device configured to move follower 174b and activation member 154b to activate and/or deactivate the vessel sealing portion 160b. In alternative embodiments, the follower 174b may be coupled to the outer jacket 146b and moving the seal actuator 170b may move the outer jacket 146b to activate and/or deactivate the vessel sealing portion 160b.

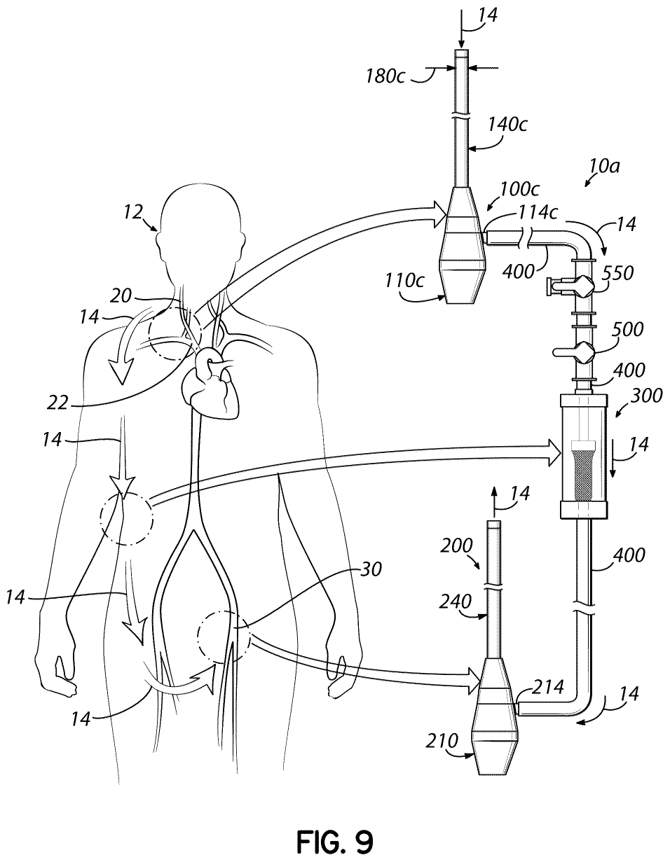

FIG. 9 illustrates an alternative embolic protection system 10a. Generally, the embolic protection system 10a is similar in construction and operation to the embolic protection system 10 described above, and the embolic protection system 10a may be substituted for other embolic protection systems, or any feature of the embolic protection system 10a may be used, in various other embodiments according to the present disclosure. Like reference numbers refer to like components. For brevity, the following description minimizes redundant description and focuses on the differences between the embolic protection system 10 and embolic protection system 10a. In this illustrative embodiment, the embolic protection system 10a includes a vessel access device 100c, a vessel return devices 200, a filter 300, tubing 400, a flow control device 500, a 3-way valve 550, and a vessel closure device 600, see FIG. 12.

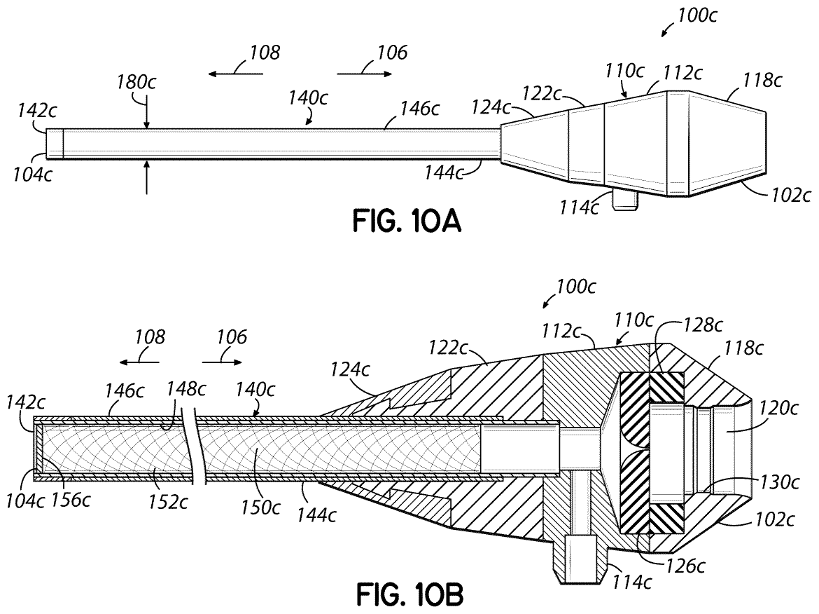

FIG. 10A is a side view of an illustrative vessel access device 100c. FIG. 10B is a sectional view detailing the construction of the vessel access device 100c. Generally, the vessel access device 100c is similar in construction and operation to the vessel access devices 100, 100a, and 100b described above and the vessel access device 100c may be substituted for other vessel access devices, or any feature of the vessel access device 100c may be used, in various other embodiments according to the present disclosure. Like reference numbers refer to like components. For brevity, the following description minimizes redundant description and focuses on the differences between the vessel access device 100c and the vessel access devices 100, 100a, and 100b.

In this illustrative embodiment, the vessel access device 100c has a proximal end portion 102c, a distal end portion 104c, a hub 110c, and a sheath 140c. For reference, arrow 106 points generally proximally and arrow 108 points generally distally. The hub 110c includes a body 112c, a side port 114c, a rear hub body 118c, a central lumen 120c, a front hub body 122c, a strain relief 124c, a dilator seal 126c, a compression washer 128c, and a lock 130c. The sheath 140c has a distal end portion 142c and a proximal end portion 144c. The sheath 140c also includes an outer jacket 146c, an inner liner 148c, a reinforcing portion 150c, a central lumen 152c, and one or more radiopaque markers 156c.

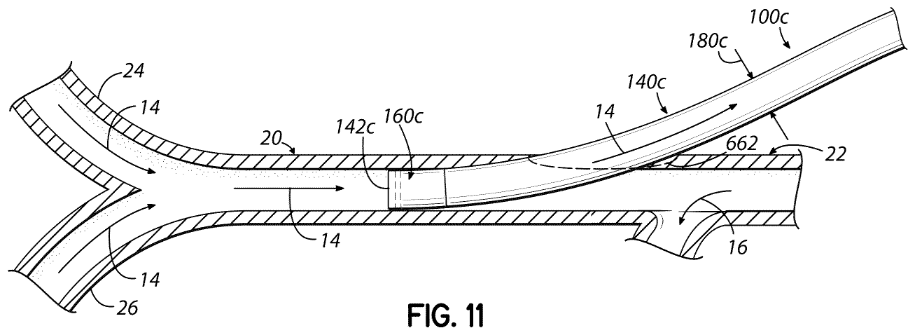

FIGS. 9 and 11 illustrate a method of percutaneously accessing blood vessels and establishing a retrograde blood flow to provide embolic protection while another medical procedure is performed. In this example, the blood vessels being accessed for establishing a retrograde blood flow are a common carotid artery 20 and a femoral vein 30. FIG. 11 depicts the vessel access device 100c percutaneously inserted into the common carotid artery 20 with the sheath 140c aligned with the lumen of the common carotid artery 20. In this alternative embodiment, the vessel access device 100c is inserted into the common carotid artery 20. The sheath 140c of the vessel access device 100c is aligned with the lumen of the common carotid artery 20. The outer diameter 180c of the sheath 140c is dimensionally similar to the inner diameter of the lumen of the common carotid artery 20. Due to the similarity of the outer diameter of the sheath 140c and the inner diameter of the lumen of the common carotid artery 20, retrograde blood flow is established without the need for additional sealing of the vessel access device 100c against the inside of the common carotid artery 20.

Devices and methods for percutaneously closing an opening in a blood vessel are described herein. FIG. 12 is a perspective view of an illustrative vessel closure device 600 configured to percutaneously close and seal a puncture or opening in a blood vessel, such as a carotid artery or a femoral vein for example. In this illustrative embodiment, the vessel closure device 600 is configured to be used with the vessel access device 100. In some embodiments, the vessel closure device 600 is configured to be used with the vessel return device 200. The vessel closure device 600 allows for a single-suture closure of a blood vessel, such as a common carotid artery 20 and/or a femoral vein 30 for example, via a minimally invasive percutaneous approach.

The vessel closure device 600 includes a handle 612 at a proximal end portion and a suturing mechanism 614 at a distal end portion. For reference, arrow 602 points generally proximally and arrow 604 points generally distally. As used herein, “suture”, “suturing” and similar forms of these terms mean any flexible tensile element or member regardless of form or material and suitable for approximating tissue. As used herein, “tensile member” may be a mono-filament suture, a multi-filament suture, a metallic suture, or any other suitable tensile member. An elongate shaft 616 extends between the handle 612 and the suturing mechanism 614. A strain relief 623 is fixed generally between the relatively rigid handle 612 and the more flexible shaft 616 to distribute forces more evenly between the handle 612 and the shaft 616.

The handle 612 includes one or more actuating mechanisms necessary for operating the suturing mechanism 614 and one or more suturing needles described herein. In this exemplary embodiment, the actuating mechanisms include a sliding suturing mechanism actuator 620 that operates the suturing mechanism 614 and a plunger style needle actuator 622 that operates the suturing needles. While the actuators are shown and described as manually driven, one or more of the actuators may be motorized, mechanically leveraged, or assisted in other manners.

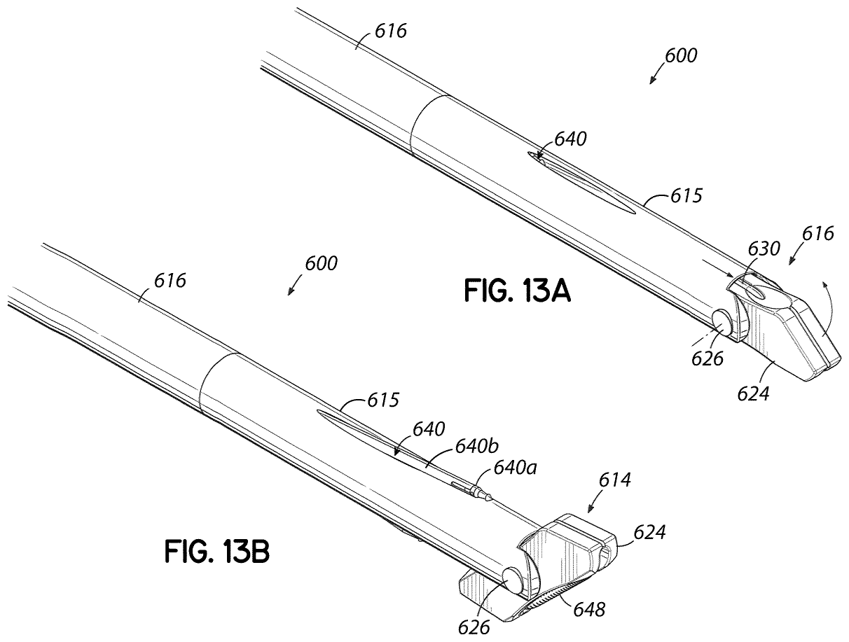

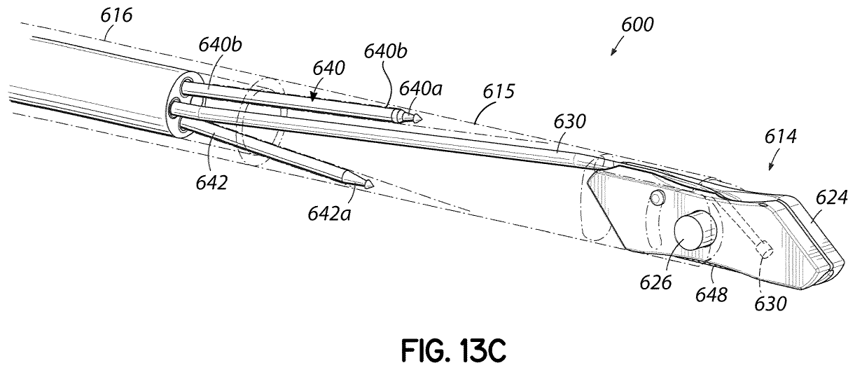

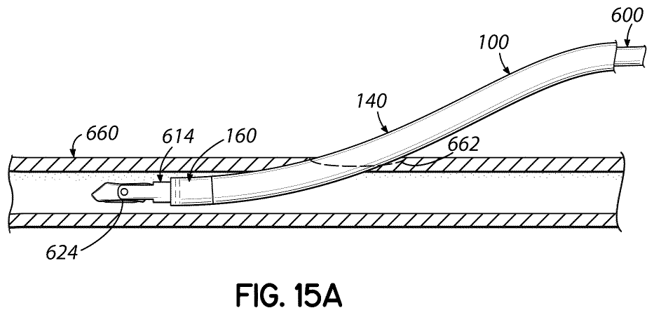

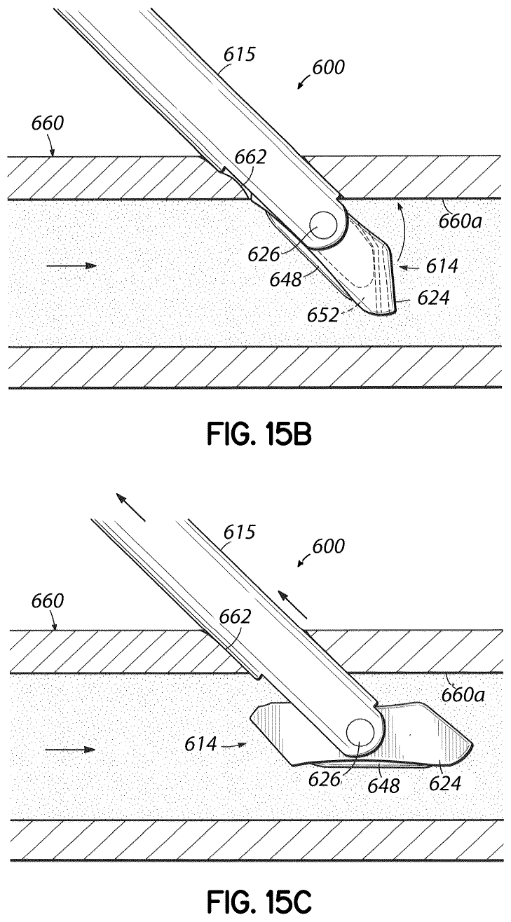

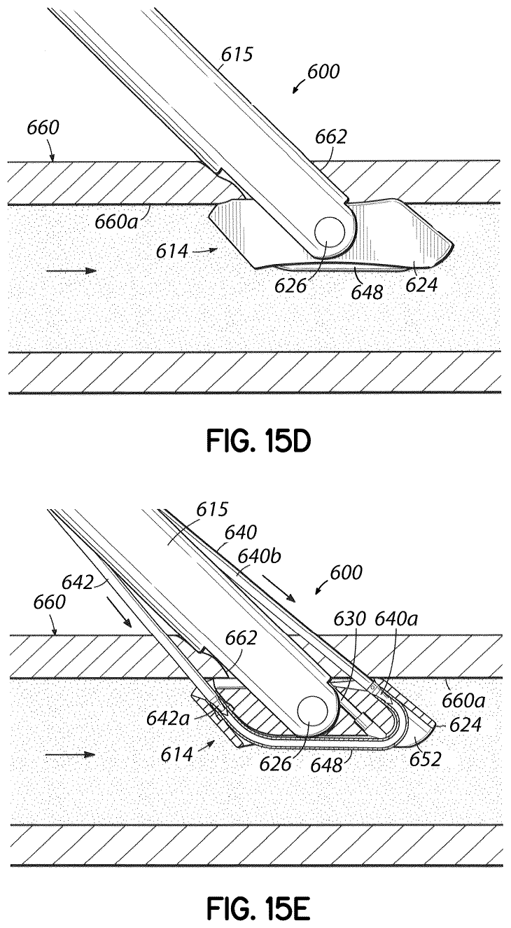

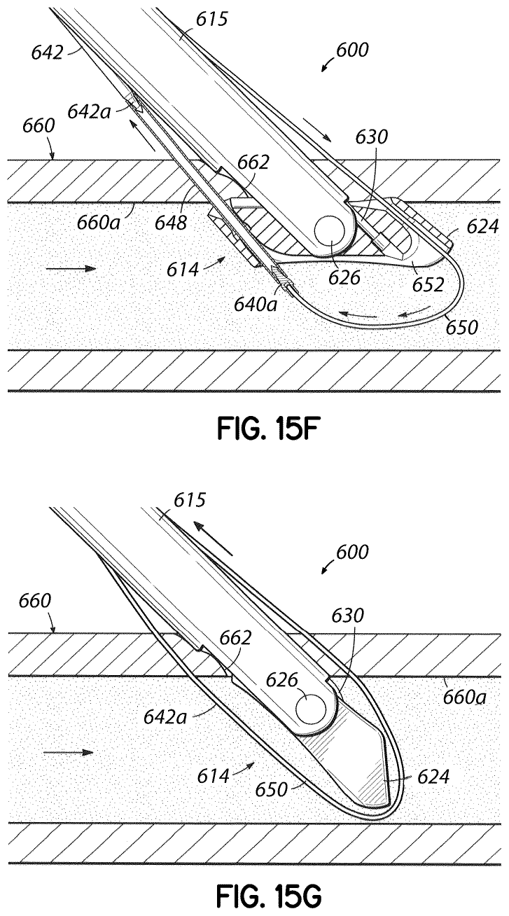

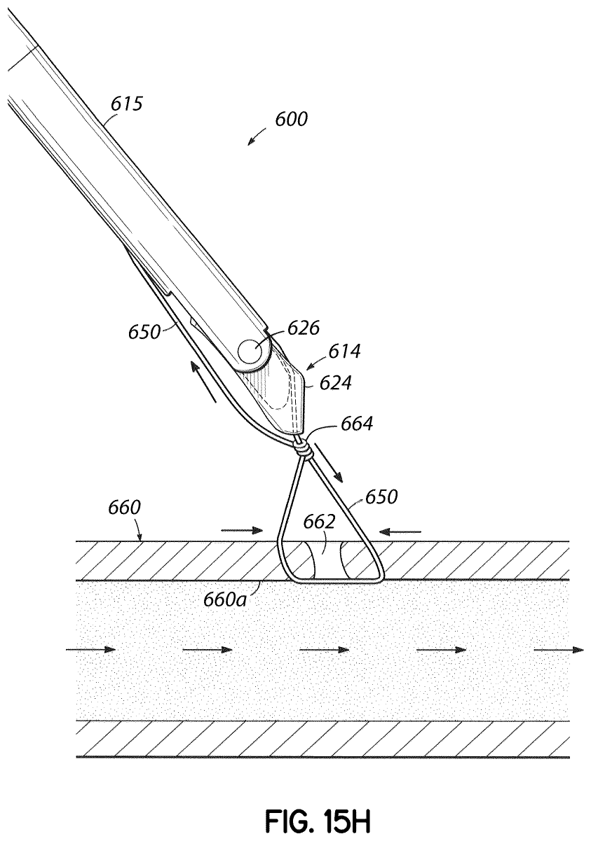

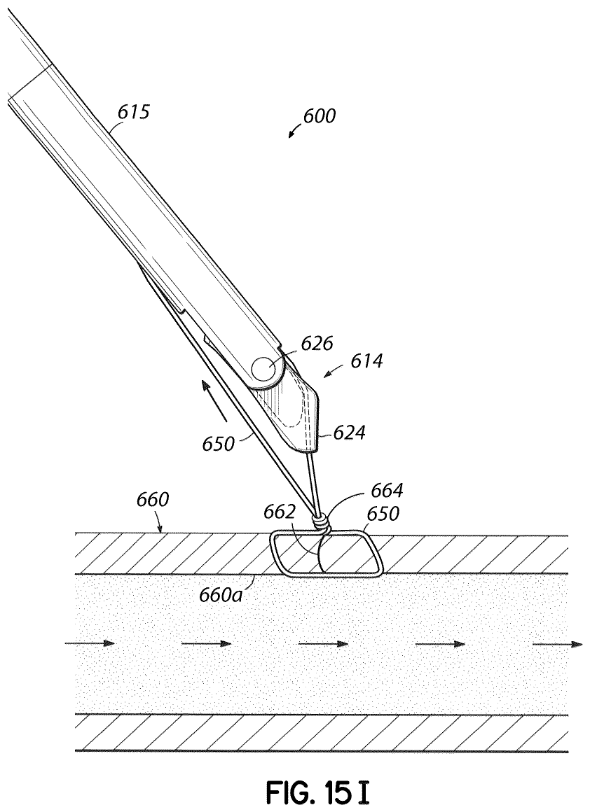

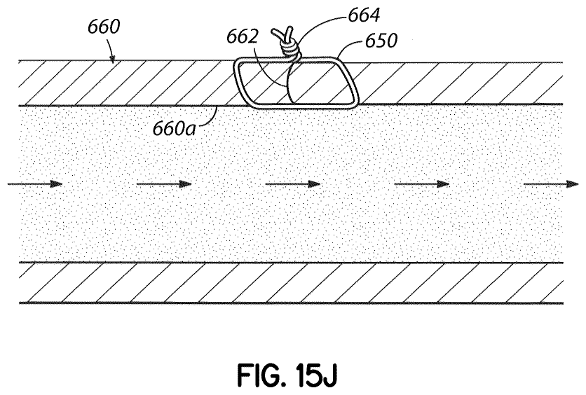

FIGS. 13A and 13B are perspective views of the distal end of the shaft 616 and the suturing mechanism 614. FIG. 13C is a perspective view of the distal end of the shaft 616 and the suturing mechanism 614 with portions of the shaft 616 and the suturing mechanism 614 shown in phantom. The suturing mechanism 614 includes a needle guide 615 and a pivotal element 624. The pivotal element 624 is secured to the distal end of the needle guide 615 by a pivot 626 that allows the pivotal element 624 to pivot from an orientation, shown in FIG. 13A, that generally aligns the lengthwise extent of the pivotal element 624 with the central axis of the shaft 616, to an orientation, shown in FIG. 13B, that is transverse to the central axis of the shaft 616. The aligned orientation of the pivotal element 624 shown in FIG. 13A is an insertion and removal orientation. The transverse orientation of the pivotal element 624 shown in FIG. 13B is a deployed orientation that facilitates a suturing procedure from within a blood vessel. The suturing mechanism actuator 620, see FIG. 12, is coupled to the pivotal element 624 by an actuating wire 630. The pivotal element 624 includes an elongate flexible coupling member 648 in passage 652, see FIG. 15E

Referring to FIGS. 12 and 13C, the distal end of the vessel closure device 600 includes a first needle 640 and a second needle 642. The first needle 640 is a two-piece needle which has a first needle tip 640a that is detachable from a first needle body 640b by way of a friction fit, for example. An elongate suture or tensile member 650 is coupled to the first needle tip 640a, see FIG. 15F. The second needle 642 has an integral (i.e., fixed) second needle tip 642a. The word “integral” here encompasses fully integral constructions as well as constructions in which the second needle tip 642a is fixedly (i.e., not “removably”) coupled to the second needle 642. The shaft 616 may be a multi lumen flexible catheter in which one of the lumens carries the actuator wire 630, two of the lumens carry the first and second needles 640, 642, and a fourth lumen may be used as a blood port for indicating to the user when the distal end of the vessel closure device 600 has entered a blood vessel. The first and second needles 640, 642 are coupled to the needle actuator 622. The first and second needles 640, 642 are deployed or moved distally by depressing the needle actuator 622 in a forward or distal direction. Pulling the needle actuator 622 in a proximal direction retracts the first and second needles 640, 642 in a proximal direction.

Referring to FIGS. 12, 13A-13C, when the suturing mechanism actuator 620 is in a neutral position, the pivotal element 624 is in the insertion and removal orientation shown in FIG. 13A. Pulling or moving the suturing mechanism actuator 620 in a proximal direction pulls the actuating wire 630 and activates the suturing mechanism 614 and pivot the pivotal element 624 from the insertion and removal orientation shown in FIG. 13A into the deployed orientation shown in FIG. 13B. The suturing mechanism actuator 620 may be mechanically locked into position, such as by being moved within a “J” or “L” slot in the handle 612, for example. Releasing or moving the suturing mechanism actuator 620 in a forward or distal direction will allow the pivotal element 624 to pivot from the deployed orientation shown in FIG. 13B to the insertion and removal orientation shown in FIG. 13A.

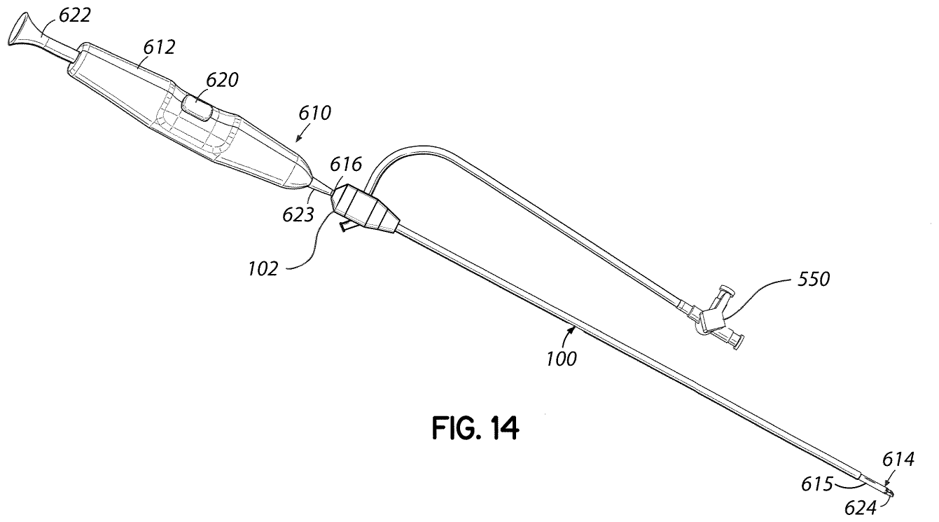

FIG. 14 shows the distal end and the suturing mechanism 614 of the vessel closure device 600 inserted through the vessel access device 100. Depth markers or indicators on the shaft 616 of the vessel closure device 600 provide visual feedback to the user as to how far to insert the vessel closure device 600 into the vessel access device 100. The dilator seal 126, see FIG. 2A, seals against the shaft 616 of the vessel closure device 600 thereby minimizing or preventing blood loss.

FIGS. 15A through 15J are side views of the distal end of the shaft 616 and the suturing mechanism 614 of the vessel closure device 600. FIGS. 15A through 15J progressively illustrate additional structure and function of the vessel closure device 600 and, particularly, the distal end portion and the suturing mechanism 614 closing and sealing a puncture or opening 662 in a wall of the blood vessel 660, such as a common carotid artery 20 or a femoral vein 30 for example, see FIG. 1.

In this illustrative example, prior employing the vessel closure device 600, the vessel sealing portion 160 of the vessel access device 100 is deflated to restore anterograde blood flow in the common carotid artery 20, see FIG. 1. Referring to FIG. 15A, the distal end of the needle guide 615, with the pivotal element 624 in the insertion and removal orientation, is inserted into the vessel access device 100 and into the blood vessel 660. The vessel closure device 600 is exchanged internally with the vessel access device 100 while within the blood vessel 660. The vessel closure device 600 is inserted into the blood vessel 660 prior to the removal of the vessel access device 100. Depth markers or indicators on the shaft 616 of the vessel closure device 600 provide visual feedback to the user as to how far to insert the vessel closure device 600 into the vessel access device 100. The vessel access device 100 is withdrawn from the blood vessel 660. The pivotal element 624 is rotated within the blood vessel 660 from the insertion and removal orientation shown in FIG. 15B into the deployed orientation shown in FIG. 15C. As shown in FIG. 15D, the pivotal element 624 is pulled proximally against the interior of the vessel wall 660a. Referring to FIG. 15E, the first and second needles 640, 642 are deployed or moved distally through the vessel wall 660a adjacent to the opening 662 and the first and second needle tips 640a, 642a are coupled to the ends of the coupling member 648. Referring to FIG. 15F, the first needle body 640b and the second needle 642 are retracted proximally. The first needle tip 640a is detached from the first needle body 640b. As the second needle 642 is pulled in a proximal direction, the second needle 642 will pull the coupling member 648 through the passage 652 in the pivotal element 624. The second needle 642 pulls the flexible coupling member 648, first needle tip 640a, and tensile member 650 through the vessel wall 660a adjacent to the opening 662.

Referring to FIG. 15G, the pivotal element 624 is actuated into its insertion and removal orientation to allow the vessel closure device 600 and, specifically, the suturing mechanism 614 to be pulled proximally and removed from the opening 662 in the blood vessel 660. As shown in FIG. 15H, as the suturing mechanism 614 is removed from the opening 662, the tensile member 650 draws the opening 662 closed and a suture knot 664 is deployed. The suture knot 664 may be any desired form of knot suitable for tying off a suture, such as the type that will automatically tighten as at least one end of the tensile member 650 is pulled. In some embodiments, the suture knot 664 may be a “Tennessee Slider” knot. The knot 664 is tightened against the outside of the blood vessel 660 to close and seal the opening 662 as shown in FIG. 151 and the ends of tensile member 650 are trimmed as shown in FIG. 15J.

The method of percutaneously closing an opening 662 in a blood vessel 660 may also include percutaneously closing an opening in the femoral vein 30 using the vessel closure device 600, see FIG. 1. The method of closing the femoral vein 30 would be similar to the method described above with the principal difference being the use of the vessel return device 200 in place of the vessel access device 100.

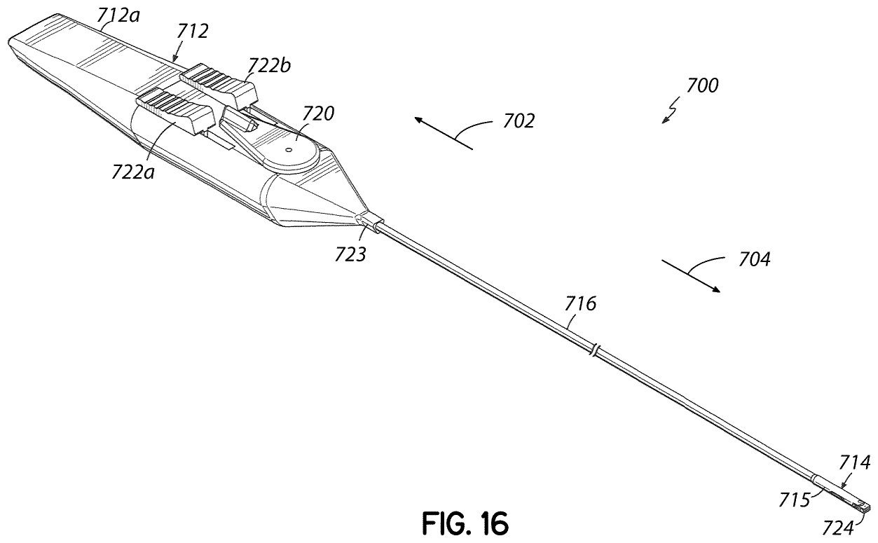

FIG. 16 is a perspective view of an alternative vessel closure device 700. In this illustrative embodiment, the vessel closure device 700 is configured to be used with the vessel access device 100. In some embodiments, the vessel closure device 700 is configured to be used with the vessel return device 200. The vessel closure device 700 allows for a single-suture closure of a blood vessel, such as a carotid artery, a femoral vein, or another blood vessel, via a minimally invasive percutaneous approach. Generally, the vessel closure device 700 is similar in construction and operation to the vessel closure device 600 described above and the vessel closure device 700 may be substituted for other vessel closure devices, or any feature of the vessel closure device 700 may be used, in various other exemplary embodiments according to the present disclosure. Like reference numbers refer to like components. For brevity, the following description minimizes redundant description and focuses on the differences between the vessel closure device 700 and the vessel closure device 600.

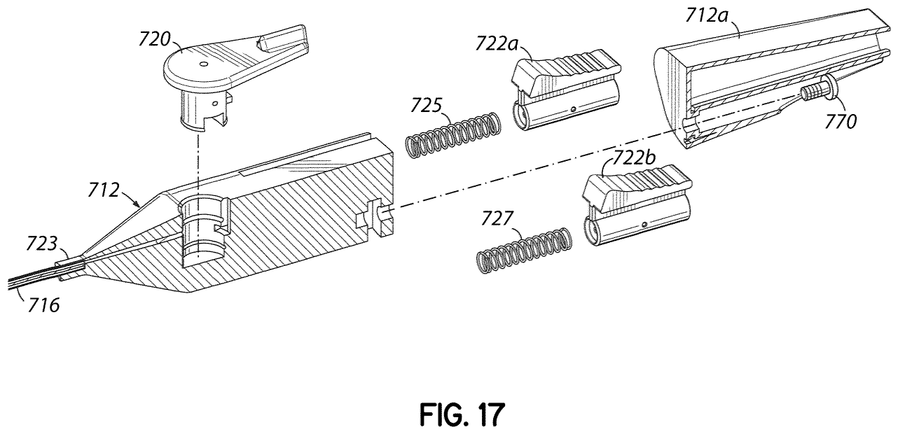

The suturing device 700 includes a handle 712 at a proximal end portion and a suturing mechanism 714 at a distal end portion. For reference, arrow 702 points generally proximally and arrow 704 points generally distally. An elongate shaft 716 extends between the handle 712 and the suturing mechanism 714. FIG. 17 is an exploded perspective view illustrating the handle 712. Referring to FIGS. 16 and 17, the handle 712 includes a pivoting, lever type suturing mechanism actuator 720 coupled to and configured to operate the suturing mechanism 714. The handle 712 also includes first and second needle actuators 722a, 722b with biasing members or coil springs 725, 727. Other forms of biasing members may be used in place of the springs 725, 727. A flexible strain relief 723 is fixed generally between the relatively rigid handle 712 and the more flexible shaft 716 to more evenly distribute forces between these components. A rotatable handle portion 712a is held to the remaining portion of the handle 712 by a fastener 770. While actuators 720, 722a, 722b are shown and described herein as manually driven, one or more of the actuators may be motorized, mechanically leveraged or assisted in other manners.

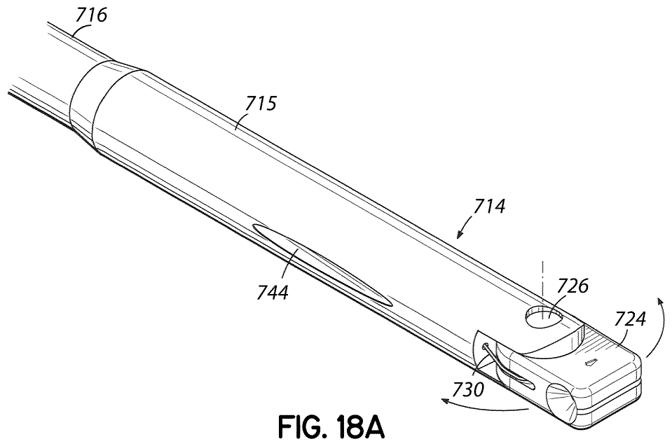

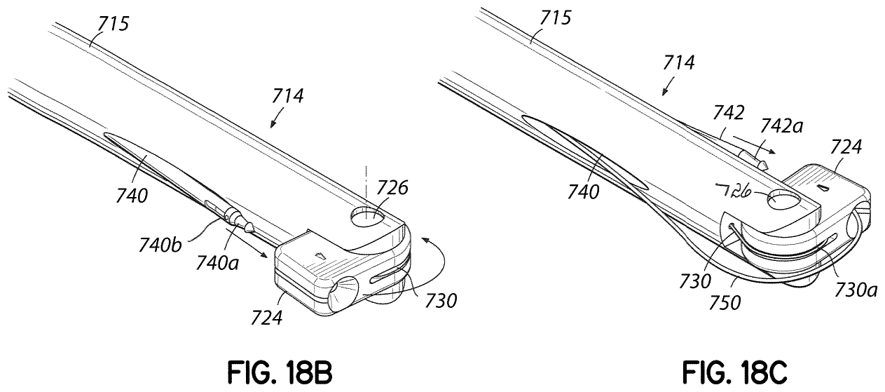

FIGS. 18A, 18B and 18C are perspective views of the distal end of the shaft 716 and the suturing mechanism 714. The suturing mechanism 714 includes a needle guide 715 and a pivotal element 724 secured to the distal end of the needle guide 715 by a pivot 726. FIG. 18A shows pivotal element 724 in an insertion and removal orientation that generally aligns the lengthwise extent of the pivotal element 724 with the lengthwise axes of the needle guide 715 and the shaft 716. FIGS. 18B and 18C show the pivotal element 724 in respective first and second deployed orientations in which the lengthwise extent of the pivotal element 724 is transverse to the lengthwise axes of the needle guide 715 and the shaft 716. This pivot 726 allows the pivotal element 724 to pivot or rotate between the insertion and removal orientation and the first and second deployed orientations. As described below, the first and second deployed orientations of the pivotal element 724 facilitate a suturing procedure to close a puncture or opening in a blood vessel. Rotating or pivoting the suturing mechanism actuator 720 on the handle 712 moves the pivotal element 724 between the insertion and removal orientation and the first and second deployed orientations.

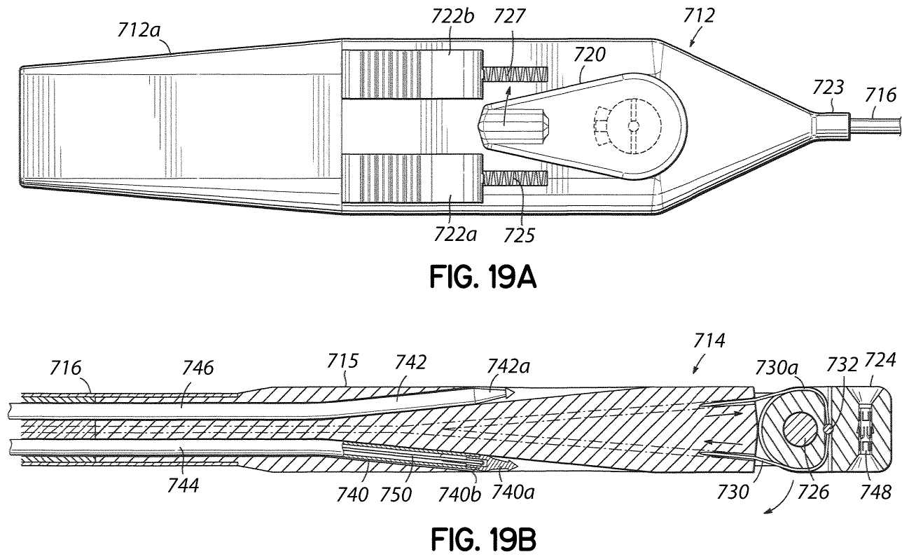

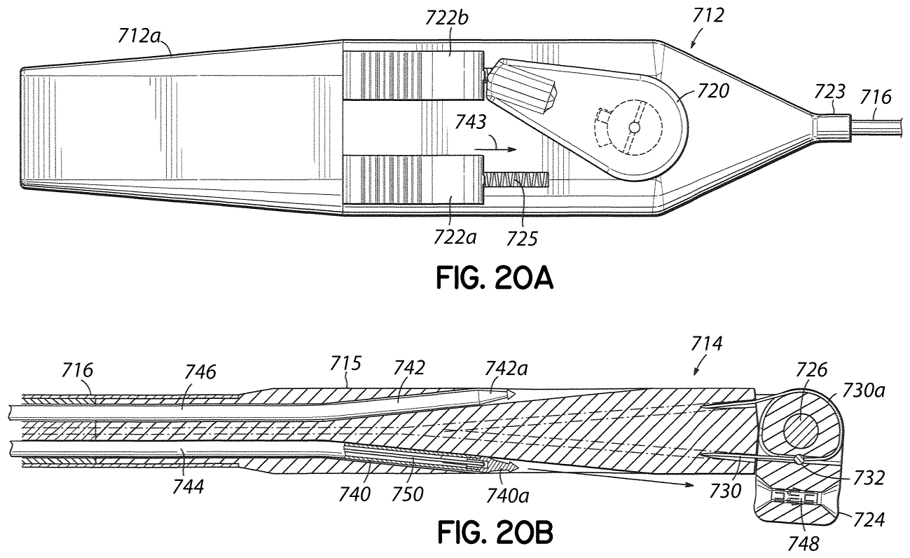

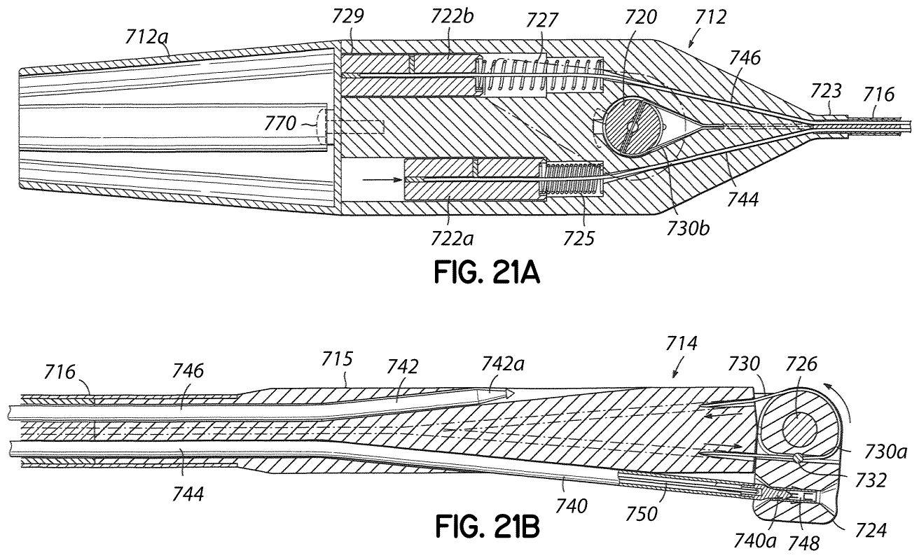

FIG. 19A is a top view of the handle 712 of the vessel closure device 700 and FIG. 19B is a sectional view of the suturing mechanism 714 in an insertion and removal orientation. FIG. 20A is a top view of the handle 712 of the vessel closure device 700 and FIG. 20B is a sectional view of the suturing mechanism 714 in a first deployed orientation. FIG. 21A is a sectional view of the handle 712 of the vessel closure device 700 and FIG. 21B is a sectional view of the suturing mechanism 714 in a first deployed orientation.

Referring to FIGS. 19A and 19B, the distal end portion of the vessel closure device 700 includes a first needle 740 and a second needle 742 which are either fully integral with or fixedly coupled to respective wires 744, 746. The first needle 740 is a two-piece needle which has a first needle tip 740a detachable from a first needle body 740b by way of a friction fit, for example. An elongate suture or tensile member 750 is coupled to the first needle tip 740a. The second needle 742 includes an integral second needle tip 742a. The first and second needles 740, 742 may be fully integral with or fixedly coupled to respective wires 744, 746. Elements 740, 742, 744, and 746 may be, for example, solid wire-like members or hollow (e.g., hypotubes). The wire-like or hypotube element 744, coupled to the first needle 740, is fixed to the first needle actuator 722a, while the wire or hypotube element 746, coupled to the second needle 742, is fixed to the actuator 722b.

Referring to FIGS. 21A and 21B, the suturing mechanism actuator 720 is coupled to the pivotal element 724 by an actuating wire 730. A first loop 730a of the wire 730 is fixed at a point 732 to the pivotal element 724 and a second loop 730b of the wire 730 is fixed for rotation with the suturing mechanism actuator 720. The pivotal element 724 includes a flexible coupling member 748. The shaft 716 may be a multi lumen flexible catheter in which one of the lumens carries the actuator wire 730, two of the lumens carry the first and second needles 740, 742, and a fourth lumen may be used as a blood port for indicating to the user when the distal end of the vessel closure device 700 has entered a blood vessel.

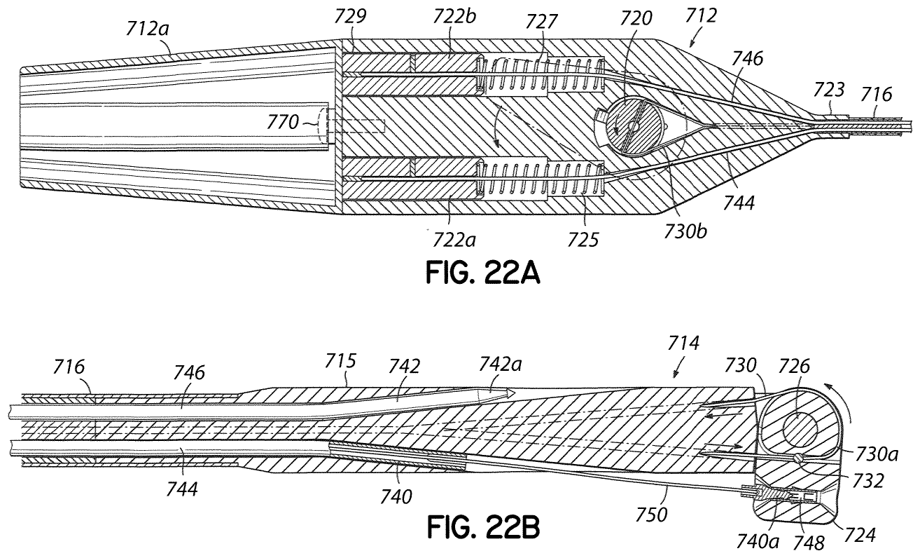

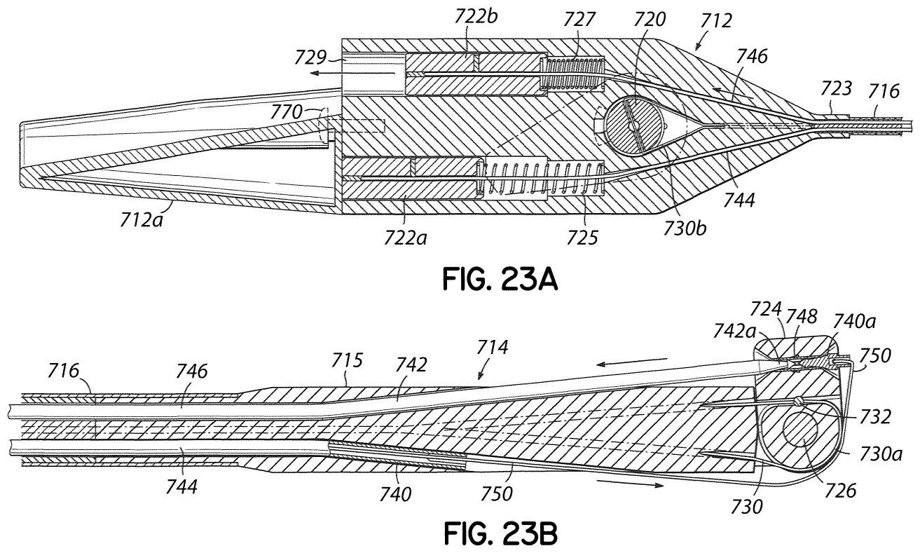

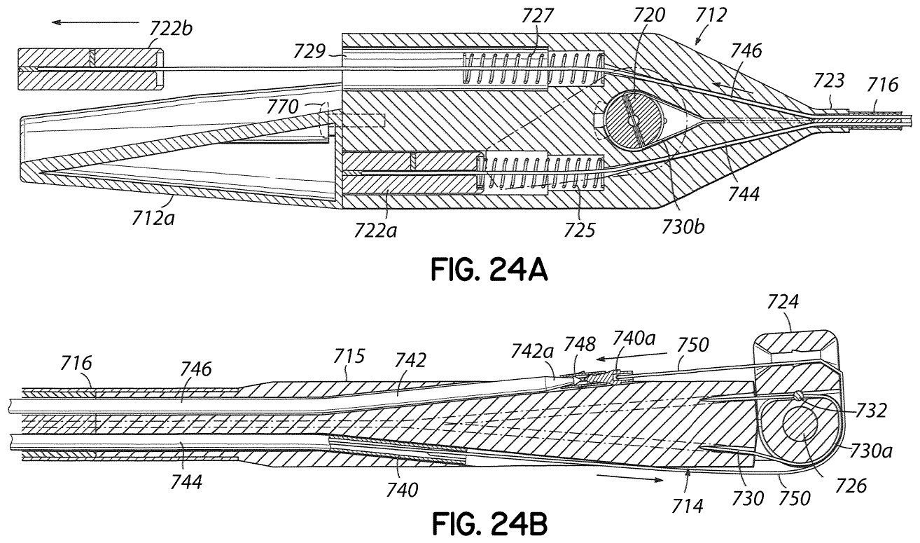

FIG. 22A is a sectional view of the handle 712 of the vessel closure device 700 and FIG. 22B is a sectional view of the suturing mechanism 714 in an first deployed orientation. FIG. 23A is a sectional view of the handle 712 of the vessel closure device 700 and FIG. 23B is a sectional view of the suturing mechanism 714 in an second deployed orientation. FIG. 24A is a sectional view of the handle 712 of the vessel closure device 700 and FIG. 24B is a sectional view of the suturing mechanism 714 in an second deployed orientation.

FIGS. 19A through 24B progressively illustrate additional structure and operation of the vessel closure device 700. As stated above, FIGS. 19A and 19B show the vessel closure device with the suturing mechanism 714 in an insertion and removal orientation. When the suturing mechanism actuator 720 is in the aligned or neutral position shown in FIG. 19A, the pivotal element 724 is in the insertion and removal orientation shown in FIG. 19B. The suturing mechanism actuator 720 is pivoted or rotated clockwise or in a first direction into the position shown in FIG. 20A to pivot or rotate the pivotal element 724 into the first deployed orientation shown in FIG. 20B. While the pivotal element 724 is in the first deployed orientation, the first needle 740 is deployed or moved in a distal direction by pushing the first needle actuator 722a in a distal direction as indicated by the arrow 743 and against the bias of the spring 725 thereby moving the first needle 740 in a distal direction toward the coupling member 748 attached to the pivotal element 724.

FIGS. 21A and 21B illustrate the positions of the first needle actuator 722a, spring 725, and first needle 740 after rotating the suturing mechanism actuator 720 and pushing the first needle actuator 722a in a distal direction. The first needle tip 740a engages and connects with a first end of coupling member 748. As shown in FIG. 20A, the suturing mechanism actuator 720 also blocks any movement of the actuator 722b, thereby preventing unintended deployment of the second needle 742. The suturing mechanism actuator 720 may be held or temporarily locked in place by detents or other suitable structure. As shown in FIGS. 22A and 22B, when released, the first needle actuator 722a is moved proximally by the expansion of the spring 725 thereby proximally retracting the needle body 740b and de-coupling the needle body 740b from the first needle tip 740a.

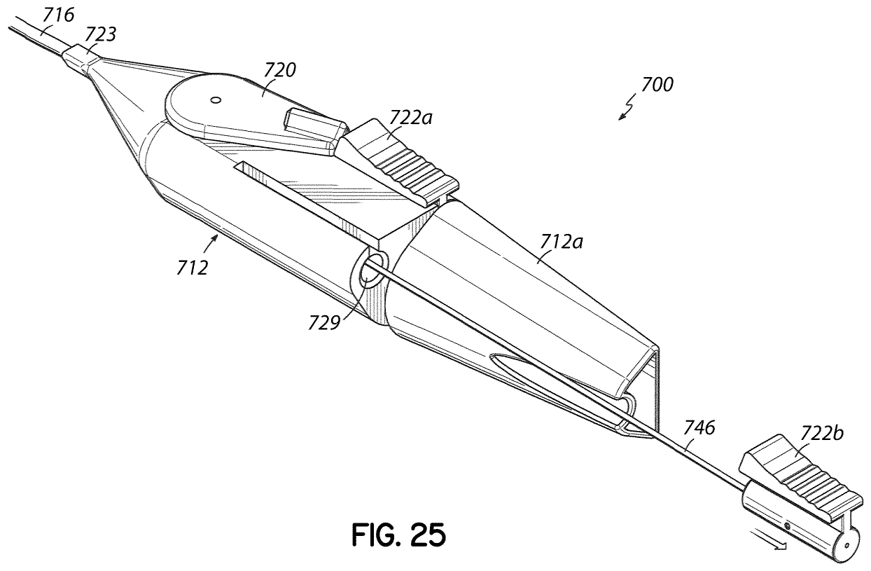

As shown in the progression of FIGS. 22A and 22B to FIGS. 23A and 23B, the suturing mechanism actuator 720 is rotated in a second or counterclockwise direction to rotate the pivotal element 724 into its second deployed orientation as shown in FIG. 23B. The actuator 722b is pushed distally against the bias of the spring 727, thereby moving the second needle 742 distally and engaging and connecting the needle second tip 742a with a second end of the coupling member 748. When the actuator 722b is released as indicated by the arrow in FIG. 23A, the expansion of the spring 727 pulls the second needle 742, including the second tip 742a, proximally and carries with it the coupling member 748 and the attached first needle tip 740a and tensile member 750 as shown in FIGS. 24A and 24B. The coupling member 748 is fixed to the pivotal element 724 with a “one-way” connection such that it may only move out from the pivotal element 724 in the direction shown in FIG. 24B. A rotatable portion 712a located at a proximal end portion of the handle 712, is rotated as further shown in FIG. 25 to expose an opening 729 allowing actuator 722b to be removed and for the second needle 742, coupling member 748, first needle tip 740a and tensile member 750 to be withdrawn in a proximal direction as shown in FIG. 24B.

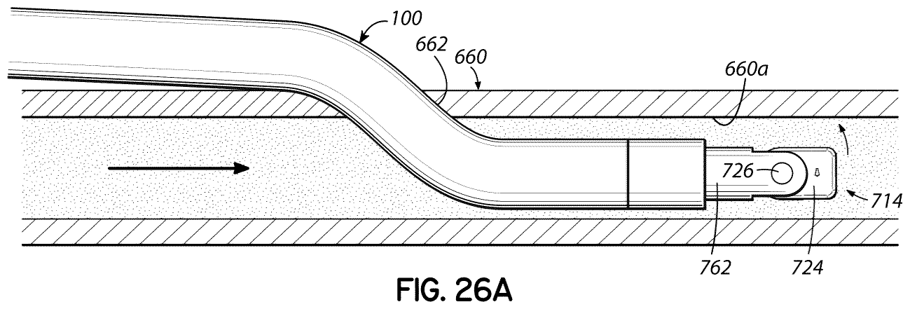

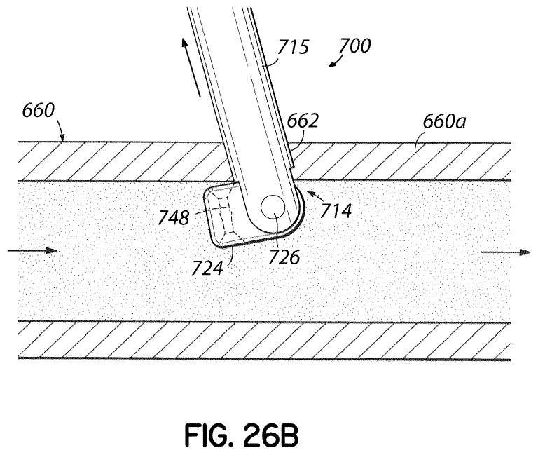

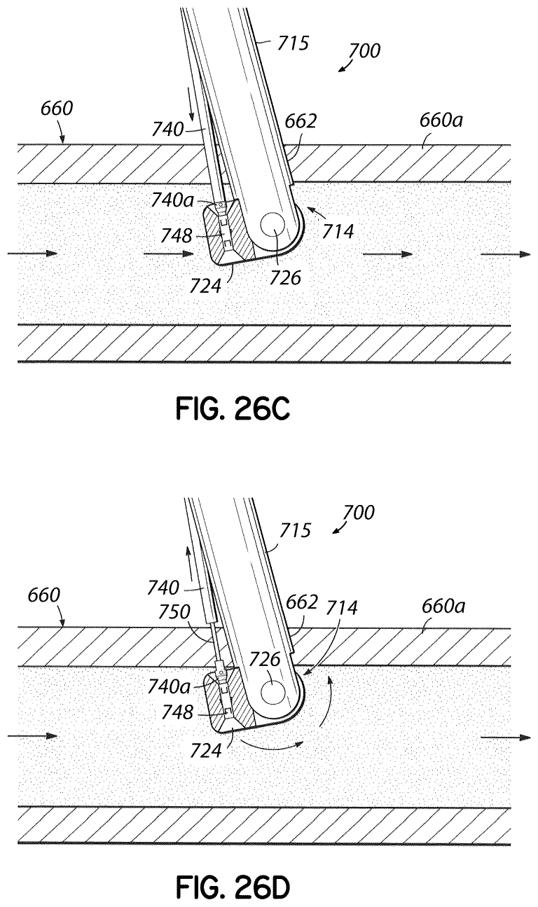

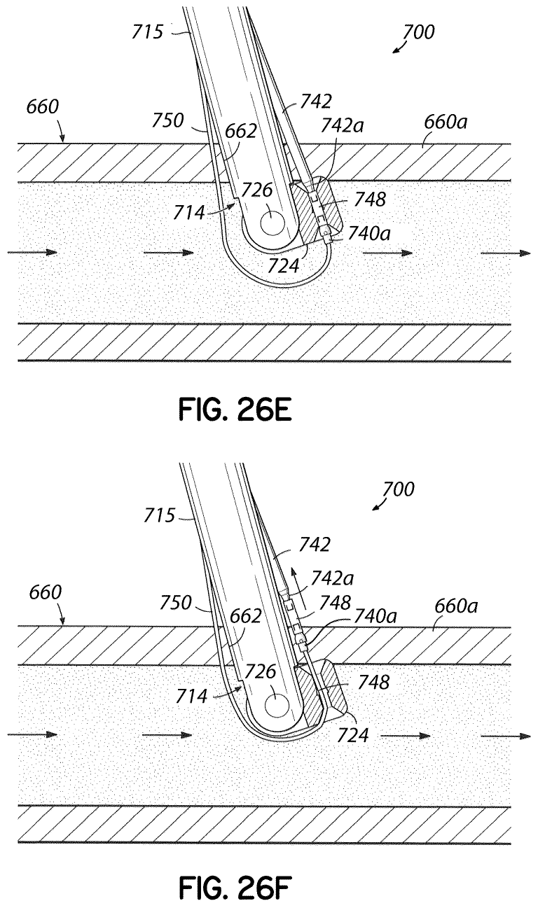

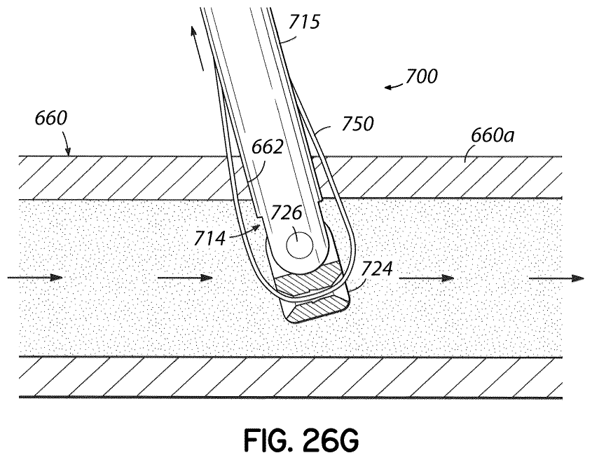

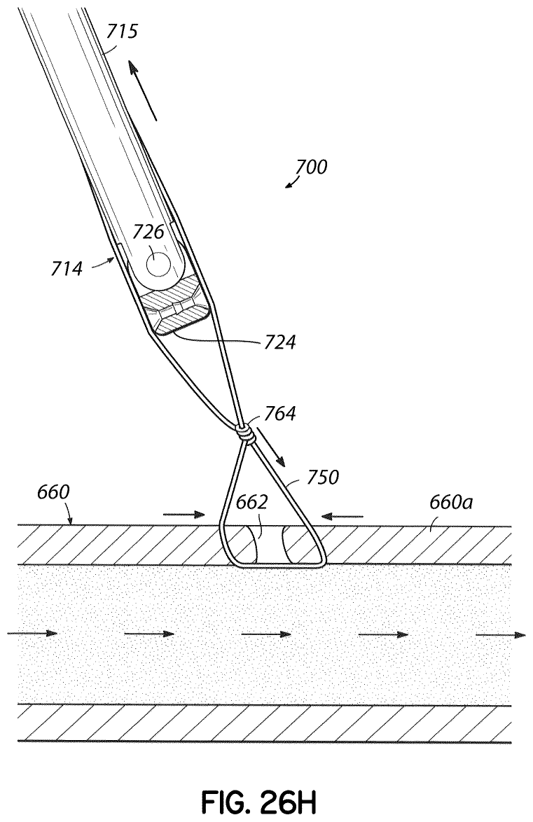

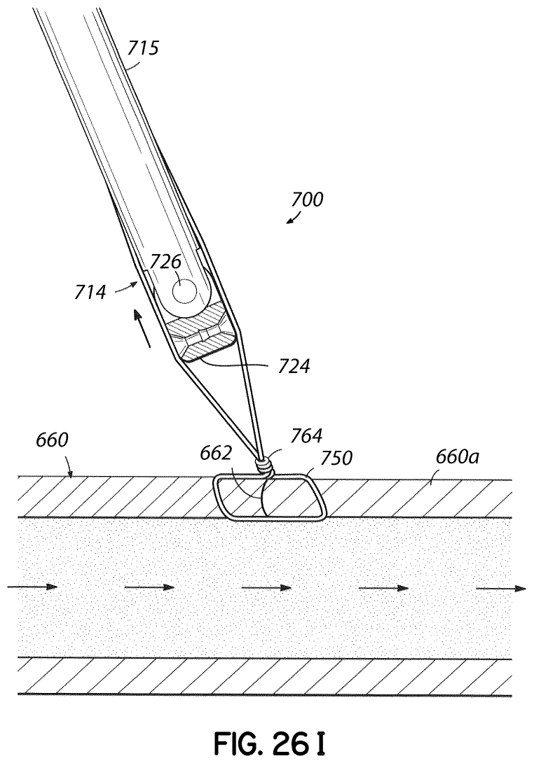

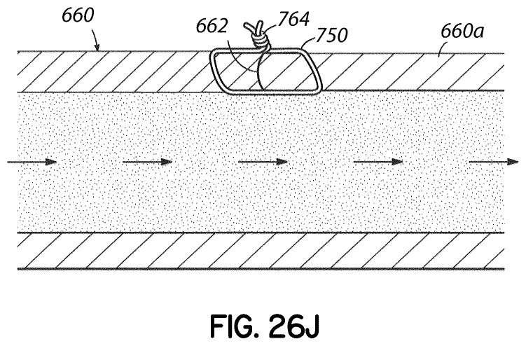

FIGS. 26A through 26J are elevation views of the distal end of the shaft 716 and the suturing mechanism 714. FIGS. 24A through 24J progressively illustrate additional structure and function of the vessel closure device 700 and, particularly, the distal end portion and the suturing mechanism 714 closing and sealing an opening 662 in a wall of the blood vessel 660, such as a carotid artery or a femoral vein for example.

Referring to FIG. 26A, the distal end of the needle guide 715, with the pivotal element 724 in the insertion and removal orientation, is inserted into the vessel access device 100 and into the blood vessel 660. The vessel closure device 700 is exchanged internally with the vessel access device 100 while within the blood vessel 660 similar to the procedure described above for the vessel closure device 600. The vessel closure device 700 is inserted into the blood vessel 660 prior to the removal of the vessel access device 100. Depth markers or indicators on the shat 716 of the vessel closure device 700 provide visual feedback to the user as to how far to insert the vessel closure device 700 into the vessel access device 100.

The suturing mechanism 714 is activated within the vessel 660 and the pivotal element 724 is pivoted into the first deployed orientation as shown in FIG. 26B. The pivotal element 724 is pulled proximally against the interior of the vessel wall 660a by slightly pulling proximally on the vessel closure device 700 and thereby the needle guide 715 as shown in FIG. 26B. The vessel access device 100 is pulled rearward or proximally along the shaft 716 while the suturing mechanism 714 remains within the vessel 660. The vessel access device 100 remains in surrounding relation to the shaft 716 for the remainder of the procedure and the vessel closure device 700 is ready for the deployment of the first and second suture needles 740, 742 and the remainder of the procedure as described herein. Once the pivotal element 724 is in this position, the first needle 740 is deployed through the vessel wall 660a adjacent the opening 662 and the first needle tip 740a connects with the first end of the coupling member 748 as shown in FIG. 26C. The first needle body 740b is retracted proximally and the first needle tip 740a is detached from the first needle body 740b while leaving the tensile member 750 fixed to the first needle tip 740a and the first needle tip 740a coupled to the coupling member 748, see FIG. 26D.

As illustrated in FIGS. 26D and 26E, the pivotal element 724 is rotated approximately 190° into the second deployed orientation. The second needle 742 is moved distally through the vessel wall 660a adjacent the opening 662 to connect the second needle tip 742a with the second end of the coupling member 748. As shown in FIG. 26F, the second needle 742, coupling member 748, first needle tip 740a and tensile member 750 are pulled in a proximal direction through the vessel wall 660a. As shown in FIG. 26G, the pivotal element 724 is rotated into the insertion and removal orientation allowing the vessel closure device 700 and, particularly, the needle guide 715 and the suturing mechanism 714 to be pulled proximally and removed from the blood vessel 660, see FIG. 26H. As further shown in FIG. 24H, a suture knot 764 is deployed. The suture knot 764 may be any desired form of knot suitable for tying off a suture, such as the type that will automatically tighten as at least one end of the tensile member 750 is pulled. The knot 764 is tightened against the outside of the vessel 660 to close and seal the opening 662 as shown in FIG. 241 and the ends of the tensile member 750 are trimmed as shown in FIG. 24J.

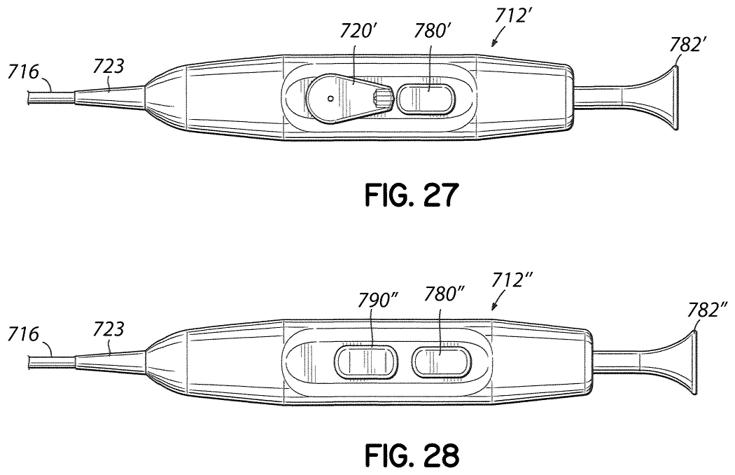

FIG. 27 is a top view of an illustrative alternative vessel closure device handle 712′. Generally, the handle 712′ is similar in construction and operation to the handle 712 described above and the handle 712′ may be substituted for other handles, or any feature of the handle 712′ may be used, in various other embodiments according to the present disclosure. Like reference numbers refer to like components. For brevity, the following description minimizes redundant description and focuses on the differences between the handle 712 and the handle 712′.

The handle 712′ includes a rotatable suturing mechanism actuator 720′ which operates similar to the suturing mechanism actuator 720 described above. The suturing mechanism actuator 720′ is rotated to rotate the pivotal element 724 from the insertion and removal orientation, see FIG. 18A, to a first deployed orientation, see FIG. 18B. The user moves the first needle 740 distally to engage the pivotal element 724, see FIG. 18B, by sliding a button 780′ forward or distally. Similar to the handle 712, a spring return mechanism (not shown) is coupled with the button 780′ and first needle 740. The suturing mechanism actuator 720′ is rotated to pivot the element 724 to the second deployed orientation, see FIG. 18C. The second needle 742 is moved distally by depressing a plunger 782′ in a distal or forward direction, see FIG. 18C. The plunger 782′ may also be coupled to a spring biased return mechanism (not shown) and may be fully removed in a proximal direction to remove the second needle 742 and tensile member 750 as previously described. The remainder of the handle 712′ operation is similar to the described operation of handle 712.

FIG. 28 is a top view of an illustrative alternative vessel closure device handle 712″. Generally, the handle 712″ is similar in construction and operation to the handles 712 and 712′ described above and the handle 712″ may be substituted for other handles, or any feature of the handle 712″ may be used, in various other embodiments according to the present disclosure. Like reference numbers refer to like components. For brevity, the following description minimizes redundant description and focuses on the differences between the handles 712 and 712′ and the handle 712″.

The handle 712″ includes actuators in the form of slide buttons 780″, 790″ and a plunger 782″. The user moves the slide button 790″ forward or distally to rotate the pivotal element 724 from the insertion and removal orientation, see FIG. 18A, to a first deployed orientation, see FIG. 18B. The user moves or slides a button 780″ forward or distally to move the first needle 740 distally to engage the pivotal element 724, see FIG. 18B. A spring return mechanism (not shown) is coupled with the button 780″ and the first needle 740. The user moves the slide button 790″ rearwardly or proximally to pivot the element 724 to its second, transverse orientation, see FIG. 18C. The second needle 742 is moved distally by depressing the plunger 782″ in a distal or forward direction. The plunger 782″ may also be coupled to a spring biased return mechanism (not shown) and may be fully removed in a proximal direction to remove the second needle 742 and tensile member 750 as previously described. The remainder of the handle 712″ operation is similar to the described operation of handles 712 and 712′.

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination within and between the various embodiments. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of the general inventive concept.

What is claimed is:

1. An embolic protection system, the embolic protection system comprising:
a vessel access device configured to percutaneously access an interior of a first vessel;
a vessel return device configured to access an interior of a second vessel;
a filter configured to collect emboli;
tubing configured to couple the vessel access device, the vessel return device, and the filter; and
a vessel closure device configured to percutaneously close and seal an opening in the first vessel, the vessel closure device including:
a proximal end including a handle with a needle actuator,
an elongate shaft coupled to the handle,
a suturing mechanism coupled to a distal end of the elongate shaft, the suturing mechanism including a pivotal element configured to be activated within the first vessel,
first and second needles associated with the suturing mechanism, the first and second needles are configured to be directed through a first vessel wall adjacent to the opening in the first vessel, and at least one of the first and second needles is coupled to the needle actuator and configured to be directed through the first vessel wall with the needle actuator,
a tensile member coupled to the first needle, and
a coupling member associated with the suturing mechanism, the coupling member having a first coupling member end configured to be coupled to the first needle and a second coupling member end configured to be coupled to the second needle, and the coupling member is configured to pull the tensile member through the first vessel wall to close and seal the opening in the first vessel;
wherein the embolic protection system is configured to direct blood flow from the first vessel, through the vessel access device, the filter, and the vessel return device, and into the second vessel.

2. The embolic protection system of claim 1, wherein the vessel closure device is configured to access the interior of the first vessel through the vessel access device.

3. The embolic protection system of claim 1, further comprising a flow control device configured to be coupled to the vessel access device, the vessel return device, and the filter with the tubing;
wherein the flow control device is configured to control the blood flow from the first vessel to the second vessel.

4. The embolic protection system of claim 1, wherein the vessel access device includes a vessel sealing portion configured to seal the vessel access device at least partially against an inside of the first vessel when the vessel sealing portion is activated.

5. The embolic protection system of claim 4, wherein the vessel sealing portion comprises an expandable element;
wherein activating the vessel sealing portion includes expanding the expandable element of the vessel sealing portion thereby at least partially sealing the vessel access device against the inside of the first vessel.

6. The embolic protection system of claim 5, wherein the expandable element comprises a balloon and inflating the balloon seals the vessel access device at least partially against the inside of the first vessel.

7. The embolic protection system of claim 4, wherein the vessel sealing portion comprises a flexible material and activating the vessel sealing portion includes compressing the flexible material to radially expand the flexible material thereby sealing the vessel access device at least partially against the inside of the first vessel.

8. The embolic protection system of claim 4, wherein the vessel sealing portion comprises a pre-formed structure, and
wherein the pre-formed structure is in a deactivated or compressed state when the sealing portion is positioned inside a distal end portion of the vessel access device and positioning the pre-formed structure outside the distal end portion of the vessel access device activates the vessel sealing portion and allows the pre-formed structure to expand radially thereby sealing the vessel access device at least partially against the inside of the first vessel.

9. The embolic protection system of claim 8, wherein the pre-formed structure comprises a wire structure.

10. The embolic protection system of claim 9, wherein the wire structure includes at least one of a film or a coating that at least partially covers the wire structure to prevent blood flow through the wire structure.

11. The embolic protection system of claim 4, wherein the vessel access device further comprises a seal actuator configured to activate and/or deactivate the vessel sealing portion.

12. The embolic protection system of claim 11, wherein moving the seal actuator axially activates and/or deactivates the vessel sealing portion.

13. The embolic protection system of claim 1, wherein the vessel access device comprises an introducer sheath.

14. The embolic protection system of claim 1, wherein the vessel return device comprises an introducer sheath.

15. The embolic protection system of claim 1, wherein at least one of the vessel access device or the vessel return device includes a radiopaque marker.

16. The embolic protection system of claim 1, wherein the vessel closure device is configured to percutaneously close and seal an opening in the second vessel.

17. The embolic protection system of claim 16, wherein the vessel closure device is configured to access the interior of the second vessel through the vessel return device.

18. The embolic protection system of claim 1, wherein the vessel closure device further comprises a suturing mechanism actuator coupled to the suturing mechanism for moving the suturing mechanism into an activated orientation;
wherein the pivotal element is configured to be moved into a deployed orientation within the vessel by the suturing mechanism actuator.

19. The embolic protection system of claim 1, wherein the coupling member comprises a flexible coupling member.

20. The embolic protection system of claim 1, wherein the first needle includes a needle body and a detachable tip coupled to the tensile member, and the detachable tip is configured to be coupled to the first end of the coupling member.

21. The embolic protection system of claim 1, wherein the suturing mechanism is configured to be directed through the vessel access device; and
wherein the vessel access device can then be withdrawn from the opening in the first vessel with the suturing mechanism remaining in the first vessel thereby inhibiting blood from exiting the first vessel through the opening in the first vessel.

22. An embolic protection system, the embolic protection system comprising:
a vessel access device configured to percutaneously access an interior of a first vessel and including a vessel sealing portion configured to seal the vessel access device at least partially against an inside of the first vessel when the vessel sealing portion is activated;
a vessel return device configured to access an interior of a second vessel;
a filter configured to collect emboli;
a flow control device;
tubing configured to couple the vessel access device, the vessel return device, the filter, and the flow control device; and
a vessel closure device configured to percutaneously close and seal an opening in the first vessel, the vessel closure device including:
a proximal end including a handle with a needle actuator,
an elongate shaft coupled to the handle,
a suturing mechanism coupled to a distal end of the elongate shaft, the suturing mechanism including a pivotal element configured to be activated within the first vessel,
first and second needles associated with the suturing mechanism, the first and second needles are configured to be directed through a first vessel wall adjacent to the opening in the first vessel, and at least one of the first and second needles is coupled to the needle actuator and configured to be directed through the first vessel wall with the needle actuator,
a tensile member coupled to the first needle, and
a coupling member associated with the suturing mechanism, the coupling member having a first coupling member end configured to be coupled to the first needle and a second coupling member end configured to be coupled to the second needle, and the coupling member is configured to pull the tensile member through the first vessel wall to close and seal the opening in the first vessel;
wherein the embolic protection system is configured to direct a blood flow from the first vessel, through the vessel access device, the filter, the flow control device, and the vessel return device, and into the second vessel, and the flow control device is configured to control the blood flow from the first vessel to the second vessel.

23. The embolic protection system of claim 22, wherein the vessel closure device is configured to access the interior of the first vessel through the vessel access device.

24. The embolic protection system of claim 22, wherein the vessel sealing portion comprises an expandable element and activating the vessel sealing portion includes expanding the expandable element of the vessel sealing portion thereby sealing the vessel access device at least partially against the inside of the first vessel.

25. The embolic protection system of claim 22, wherein the expandable portion comprises a balloon and inflating the balloon seals the vessel access device at least partially against the inside of the first vessel.

26. The embolic protection system of claim 22, wherein the sealing portion comprises a flexible material and activating the vessel sealing portion includes compressing the flexible material to radially expand the flexible material thereby sealing the vessel access device at least partially against the inside of the first vessel.

27. The embolic protection system of claim 22, wherein the sealing portion comprises a pre-formed structure, and
wherein the pre-formed structure is in a deactivated or compressed state when the sealing portion is positioned inside a distal end portion of the vessel access device and positioning the pre-formed structure outside the distal end portion of the vessel access device activates the vessel sealing portion and allows the pre-formed structure to expand radially thereby sealing the vessel access device at least partially against the inside of the first vessel.

28. The embolic protection system of claim 27, wherein the pre-formed structure comprises a wire structure.

29. The embolic protection system of claim 28, wherein the wire structure includes at least one of a film or a coating that at least partially covers the wire structure to prevent blood flow through the wire structure.

30. The embolic protection system of claim 22, wherein the vessel access device further comprises a seal actuator configured to activate and/or deactivate the vessel sealing portion.

31. The embolic protection system of claim 30, wherein moving the seal actuator axially activates and/or deactivates the vessel sealing portion.

32. The embolic protection system of claim 22, wherein the vessel access device comprises an introducer sheath.

33. The embolic protection system of claim 22, wherein the vessel return device comprises an introducer sheath.

34. The embolic protection system of claim 22, wherein at least one of the vessel access device or the vessel return device includes a radiopaque marker.

35. The embolic protection system of claim 22, wherein the vessel closure device is configured to percutaneously close and seal an opening in the second vessel.

36. The embolic protection system of claim 22, wherein the vessel closure device is configured to access the second vessel through the vessel return device.

37. The embolic protection system of claim 22, wherein the vessel closure device further comprises a suturing mechanism actuator coupled to the suturing mechanism for moving the suturing mechanism into an activated orientation;
wherein the pivotal element is configured to be moved into a deployed orientation within the vessel by the suturing mechanism actuator.

38. The embolic protection system of claim 22, wherein the coupling member comprises a flexible coupling member.

39. The embolic protection system of claim 22, wherein the first needle includes a needle body and a detachable tip coupled to the tensile member, and the detachable tip is configured to be coupled to the first end of the coupling member.

40. The embolic protection system of claim 22, wherein the suturing mechanism is configured to be directed through the vessel access device; and
wherein the vessel access device can then be withdrawn from the opening in the first vessel with the suturing mechanism remaining in the first vessel thereby inhibiting blood from exiting the first vessel through the opening in the first vessel.

41. A method of providing embolic protection, the method comprising:
percutaneously accessing an interior of a first vessel with a vessel access device;
accessing an interior of a second vessel with a vessel return device;
coupling tubing to the vessel access device and the vessel return device;
coupling a filter to the tubing;
establishing a blood flow from the first vessel, through the vessel access device, the filter, and the vessel return device, and into the second vessel; and
percutaneously closing an opening in the first vessel by directing a suturing mechanism through the vessel access device and into the interior of the first vessel, withdrawing the vessel access device from the first vessel, activating a pivotal element within the first vessel, directing first and second needles through a first vessel wall adjacent to the opening in the first vessel, directing a tensile member through the first vessel wall adjacent to the opening in the first vessel with the first needle, coupling the first and second needles to a coupling member, withdrawing the suturing mechanism from the first vessel, pulling the coupling member and tensile member through the first vessel wall with the second needle, and tightening the tensile member to close and seal the opening in the first vessel.

42. The method of claim 41, further comprising:
coupling a flow control device to the tubing;
directing the blood flow through the flow control device; and
controlling the blood flow from the first vessel to the second vessel with the flow control device.

43. The method of claim 41, wherein the first vessel is a carotid artery.

44. The method of claim 41, wherein the second vessel is a femoral vein.

45. The method of claim 41, wherein accessing the interior of the second vessel includes percutaneously accessing the second vessel with the vessel return device.

46. The method of claim 41, further comprising sealing the vessel access device at least partially against an inside of the first vessel.

47. The method of claim 46, wherein sealing the vessel access device at least partially against an inside of the first vessel comprises expanding a vessel sealing portion of the vessel access device.

48. The method of claim 47, wherein the vessel sealing portion of the vessel access device comprises a balloon; and
the method further comprises inflating the balloon.

49. The method of claim 46, wherein sealing the vessel access device at least partially against the inside of the first vessel further comprises compressing a flexible material of a vessel sealing portion of the vessel access device to radially expand the flexible material.

50. The method of claim 46, wherein sealing the vessel access device at least partially against the inside of the first vessel further comprises activating a pre-formed structure of a sealing portion of the vessel access device allowing the pre-formed structure to expand radially.

51. The method of claim 46, wherein sealing the vessel access device at least partially against the inside of the first vessel further comprises activating a vessel sealing portion of the vessel access device with a seal actuator.

52. The method of claim 51, further comprising moving the seal actuator axially to activate the vessel sealing portion.

53. The method of claim 41, wherein withdrawing the vessel access device from the first vessel with the suturing mechanism remaining in the first vessel thereby inhibits blood from exiting the first vessel through the opening in the first vessel.

54. The method of claim 41, wherein activating the pivotal element further comprises activating the pivotal element into a deployed orientation.

55. The method of claim 41, further comprising moving the pivotal element of the suturing mechanism into a removal orientation before withdrawing the suturing mechanism from the first vessel.

56. The method of claim 41, further comprising percutaneously closing an opening in the second vessel by directing a suturing mechanism through the vessel return device and into the interior of the second vessel, withdrawing the vessel return device from the second vessel, activating a pivotal element within the second vessel, directing first and second needles through a second vessel wall adjacent to the opening in the second vessel, directing a tensile member through the second vessel wall adjacent to the opening in the second vessel with the first needle, coupling the first and second needles to a coupling member, withdrawing the suturing mechanism from the second vessel, pulling the coupling member and tensile member through the second vessel wall with the second needle, and tightening the tensile member to close and seal the opening in the second vessel.

57. The method of claim 56, wherein activating the pivotal element further comprises activating the pivotal element into a deployed orientation.

58. The method of claim 56, further comprising moving the pivotal element of the suturing mechanism into a removal orientation before withdrawing the suturing mechanism from the second vessel.

59. The method of claim 56, wherein withdrawing the vessel return device from the second vessel with the suturing mechanism remaining in the second vessel thereby inhibits blood from exiting the second vessel through the opening in the second vessel.
